(19) United States
(12) Patent Application Publication
(10) Pub. No.: US 2026/0297427 A1
(43) Pub. Date: Oct. 1, 2026

(54) Soil Conditioning Composition Comprising Micronized Mineral and Biological Components for Enhancing Water Retention and Soil Function

(71) Applicant: Gibril Gabriel Bachouchi, North York (CA)

(72) Inventor: Gibril Gabriel Bachouchi, Toronto (CA)

(73) Assignee: Kuzog France, paris (FR)

(21) Appl. No.: 19/370,895

(22) Filed: Oct. 28, 2025

Related U.S. Application Data
(60) Provisional application No. 63/780,208, filed on Mar. 29, 2025.

(51) Int. Cl. C09K 17/40; C09K 17/02; C09K 101/00; G16C 20/10

(52) U.S. Cl. CPC C09K 17/40; C09K 17/02; G16C 20/10; C09K 2101/00

(57) ABSTRACT

A soil-conditioning composition is disclosed comprising micronized smectite clay and zeolite fractions, each having a median particle size (D₅₀) between 1 μm and 50 μm, and viable arbuscular mycorrhizal fungi (AMF) propagules. The mineral components provide complementary swelling, adsorption, and ion-exchange capacities that increase soil water retention and nutrient buffering, while the biological component enhances rhizosphere colonization, root development, and soil aggregation. Synergistic interaction between the mineral and biological fractions produces higher nutrient-use efficiency and water-holding capacity relative to use of the individual components. The proportions of the components are selected according to soil-diagnostic parameters, including texture, cation-exchange capacity, and electrical conductivity, to achieve targeted conditioning performance. When reconstituted in water, the composition forms a stable suspension with an ionic strength below 50 mM and a pH of 5.5-7.5. The formulation is suitable for application in arid, semi-arid, or coarse-textured soils exhibiting low fertility and limited moisture retention.

### CROSS-REFERENCE TO RELATED APPLICATIONS

This application claims the benefit of U.S. Provisional Application No. 63/780,208, filed Mar. 29, 2025, under 35 U.S.C. 119 (e), entitled, entitled “Soil Amendment Composition and Method for Enhancing Water Retention and Nutrient Absorption, using Mycorrhizal Fungi and Liquid Clay in combination,” the entirety of which is incorporated by reference herein.

### BACKGROUND OF THE INVENTION

### Field of the Invention

The present invention relates generally to soil conditioning compositions and agronomic methods for enhancing soil functionality The invention is classified under C09K 17/14 (soil conditioning compositions). More specifically, it pertains to formulations comprising micronized sodium smectite clay (including, but not limited to, bentonite), micronized zeolite (such as clinoptilolite and chabazite), and arbuscular mycorrhizal fungi (AMF) propagules. The disclosed compositions are designed to improve soil physical properties (e.g., water retention, aggregation) and biological performance (e.g., nutrient uptake, root symbiosis), particularly in degraded, low-organic-matter, or sandy soils.

### Prior Art

Soil amendments comprising smectite clays, such as sodium bentonite, have been employed to enhance soil water-holding capacity due to their high surface area and swelling characteristics. However, conventional bentonite or zeolite products are typically provided in coarse particle sizes, that restrict surface reactivity and limit uniform integration with the soil matrix. As a result, high application rates—often in the range of tens of metric tons per hectare—are required to achieve appreciable improvements in soil moisture retention. Although nanoparticulate smectite materials may exhibit high surface reactivity in laboratory conditions, their use in agricultural contexts is generally impractical due to elevated production costs, regulatory restrictions on nanoparticle handling, increased environmental and health scrutiny. Moreover, smectite-based amendments alone fail to address issues related to nutrient retention or biological soil enhancement, thereby limiting their utility for comprehensive soil rehabilitation.

Natural zeolites, including clinoptilolite and chabazite, are valued for their high cation-exchange capacity and ability to selectively adsorb plant nutrients, offering potential benefits for reducing nutrient leaching and mitigating salt stress. Nonetheless, commercially available zeolite products are typically supplied in particle sizes exceeding 100 microns, which diminishes their water retention capacity and limits their functional reactivity in soil applications.

Arbuscular mycorrhizal fungi (AMF) have been used to promote plant nutrient and water uptake through symbiotic colonization of root systems. However, conventional AMF inoculants—formulated as dry granules or liquid suspensions—frequently exhibit poor establishment in degraded, low-organic-matter, or sandy soils, where suboptimal physical and chemical conditions impede fungal colonization. Furthermore, these formulations generally lack a protective physical matrix to shield fungal propagules during storage and application or to facilitate their effective interaction with plant roots.

Accordingly, despite the documented individual benefits of mineral and microbial amendments, there exists prior art, known to the applicant, that successfully integrates micronized mineral soil conditioners (e.g., smectite and zeolite) with viable AMF propagules in a single, synergistic composition. There remains a need for a soil conditioning formulation that (a) combines complementary functionalities related to moisture retention, nutrient exchange, and biological enhancement; (b) maintains microbial viability during production, storage, and application; and (c) delivers synergistic field performance that surpasses the additive effects of its individual components.

Under favorable soil and cropping conditions, the mineral matrix (bentonite+zeolite) forms a semi-permanent soil conditioning layer that may persist in effect for 3-5 years following a single application, reducing amendment frequency and operational costs. AMF colonization may provide residual biological benefits for up to 5-6 years.

### Technical Problem

Agricultural performance on soils characterized by sandy texture, degradation, or low organic matter is impaired by a combination of insufficient water-holding capacity, accelerated nutrient leaching, and deficient biological activity. Conventional agronomic interventions address these constraints in a disaggregated manner: smectite clay amendments improve moisture retention yet typically do not do not enhance microbial viability or function; zeolite conditioners contribute to improved cation exchange capacity (CEC) but exhibit negligible water retention; and microbial inoculants such as arbuscular mycorrhizal fungi (AMF) promote root symbiosis but lack a supporting mineral substrate to facilitate survival and colonization in adverse soil environments. Common compensatory practices—including increased fertilizer application and more frequent irrigation—temporarily mitigate these deficiencies but result in elevated input costs, greater environmental burden (e.g., eutrophication, greenhouse gas emissions), and do not address the underlying structural and biological limitations of the soil. There exists a need for an integrated, synergistic solution that concurrently addresses water retention, nutrient efficiency, and biological regeneration.

### BACKGROUND—CONTEXT AND NEED

Escalating pressures from global freshwater scarcity, rising fertilizer costs, and increasing nutrient losses via runoff and leaching have underscored the demand for integrated soil-conditioning technologies that concurrently improve water use efficiency, nutrient retention, and biological soil function. These challenges are particularly acute in arid and semi-arid regions, where elevated soil salinity and structurally poor soils further inhibit crop nutrient uptake and water availability. While conventional synthetic soil conditioners—such as polyacrylamide-based hydrogels—offer temporary enhancement of moisture retention, they are associated with high production costs, environmental persistence, and lack of contribution to long-term (non-biodegradable synthetic polymers) soil regeneration or biological enhancement. There remains a critical unmet need for sustainable, multifunctional compositions that address both environmental constraints and agronomic performance.

### SUMMARY OF THE INVENTION

The present invention relates to a synergistic soil conditioning composition and associated methods of use, comprising: (A) micronized natural sodium smectite clay, or sodium-activated smectite clay having a high surface area (>600 m²/g) and swelling capacity sufficient to increase available water capacity by ≥20% relative to untreated soil; (B) micronized zeolite with high cation-exchange capacity to improve nutrient retention and mitigate soil salinity; and (C) arbuscular mycorrhizal fungi (AMF) propagules to promote root symbiosis and enhance biological soil function. The composition increases soil available water capacity, nutrient-use efficiency, and microbial colonization, thereby reducing irrigation requirements, lowering fertilizer input, and enabling cultivation on degraded or marginal soils.

The synergistic benefit derives from functional integration of mineral and microbial components. Smectite provides a porous matrix that buffers moisture and environmental fluctuations, supporting AMF viability. Zeolite facilitates nutrient exchange while displacing excess sodium ions in saline soils, enhancing conditions for AMF colonization. The combination of sodium smectite and zeolite produces a synergistic mineral matrix wherein bentonite enhances soil moisture retention, reduces irrigation requirements by at least 15 percent as supported by the examples herein, extending the functional residence time and ion-exchange efficiency of zeolite. Conversely, zeolite stabilizes the physical structure and ionic selectivity of the bentonite-rich matrix, mitigating sodium-induced dispersion and preserving porosity. This interaction enables a dual-phase conditioning system with superior soil-structure stability, nutrient retention, and compatibility with biological inoculants such as AMF, exceeding the sum of individual component effects as supported by the examples herein. These interactions yield emergent improvements in soil structure and fertility that exceed the additive performance of the individual components, resulting in sustained crop productivity and soil regeneration.

### BRIEF DESCRIPTION OF THE DRAWINGS

FIG. 1 is a flowchart illustrating an Adaptive Formulation Workflow. The workflow is informed by site-specific soil diagnostics, including but not limited to soil texture, cation exchange capacity (CEC), electrical conductivity (EC), organic matter content (OM), and available water capacity (AWC). Component proportions and overall quantities are selected adaptively based on soil diagnostic parameters processed through a computerized decision support system, which may utilize machine learning algorithms. The flowchart outlines decision pathways that support data-driven formulation and application strategies tailored to specific soil conditions

FIG. 2 is a schematic representation of a two-stage mixing process. The first stage comprises a high shear mixing process designed for the integration of the mineral fraction into the soil. The second stage involves a low shear mixing process for biological inoculation, thereby preserving the viability and enhancing the establishment of arbuscular mycorrhizal fungi (AMF) within the soil matrix.

FIG. 3 depicts various product format embodiments, including: (1) a pre-mixed dry micronized powder; (2) a two-part kit comprising separate mineral and AMF components; (3) a liquid concentrate; and (4) a granulated form. Each format is adaptable to specific handling, storage, and application requirements.

FIG. 4 illustrates representative application methods for the soil conditioning formulations, including but not limited to broadcast spreading, in-furrow application, and fertigation-compatible deployment.

FIG. 5 presents a summary of key agronomic outcomes observed in Example 1, including irrigation reduction, yield improvement, water-use efficiency, and nitrogen-use efficiency. The figure illustrates the combined impact of the tri-technology formulation on resource use and productivity in treated plots relative to untreated controls.

FIG. 6 is a comparative bar chart illustrating total seasonal irrigation requirements across five treatment groups: untreated control, bentonite-only, zeolite-only, bentonite+zeolite (BZ), and the full tri-technology formulation containing bentonite, zeolite, and AMF (BZA). The figure highlights the progressive reduction in irrigation volume as additional components are introduced, with the greatest savings observed in the BZA treatment.

FIG. 7 displays crop yield results (kg·m⁻²) for the same five treatment groups: control, bentonite-only, zeolite-only, BZ, and BZA. The chart demonstrates that yield increases with the addition of each component, with the BZA formulation producing the highest marketable yield, confirming synergistic performance.

FIG. 8 presents a comparative analysis of water-use efficiency and nitrogen-use efficiency across the five treatment groups. The data show improved resource-use efficiency in all amended treatments, with the most significant improvements observed in the full tri-technology formulation, reflecting the integrated benefits of the physical, chemical, and biological mechanisms of action.

### LIST OF TABLES

Table 1: Comparison of Particle Size Strategies for Soil Amendment
Table 2: Core Components of the Soil Conditioning Composition
Table 3: Adaptive Formulation Selection
Table 4: Performance Comparison-composition powder vs. Control (Example 1)
Table 5: Comparative Performance Across Five Treatments (Example 2)
Table 6: Summary of Field Performance Data
Table 7 Key limitations in the prior art and solutions Provided by the Invention

### DETAILED DESCRIPTION OF THE INVENTION

### Overview

The present disclosure relates to soil-conditioning compositions hereinafter referred to as the Tri-Technology Composition (‘TTC’), associated manufacturing processes, adaptive formulation algorithms, and agronomic application methodologies that collectively enhance soil physical, chemical, and biological properties in a manner that reduces water and fertilizer inputs by ≥15 percent in representative trials. The described embodiments incorporate synergistic interactions between mineral-based and microbial components to optimize soil functionality across varied conditions. Where applicable, reference numerals correspond to the elements identified in the accompanying figures.

### 1. Core Innovation

### 1.1 Synergistic Tri-Component System

The disclosed invention is directed to a synergistic soil-conditioning composition comprising three functional components that operate cooperatively to remediate soil degradation across physical, chemical, and biological domains. Specifically, the composition integrates:
i. Micronized smectite clay, which enhances soil water retention and aggregation through its high surface area and swelling properties.
ii. Micronized zeolite, a crystalline aluminosilicate providing ion exchange capacity and nutrient retention through its porous structure and electrostatic properties; and
iii. Arbuscular mycorrhizal fungi (AMF) propagules, which establish symbiotic associations with plant roots, improving nutrient uptake, root architecture, and biological activity within the rhizosphere.

The integration of these components results in emergent synergistic effects that surpass the additive performance of the individual materials. These effects are achieved through the complementary interaction of physical water retention, chemical nutrient buffering, and biological root enhancement.

This Synergistic Tri-Technology Integration enables adaptive formulations that can address multiple limitations in degraded or marginal soils. The formulation can be tailored based on site-specific diagnostic data to simultaneously optimize water availability, nutrient retention, and biological functionality, thereby enhancing plant productivity and soil resilience in a comprehensive manner.

### 1.2 Particle Engineering for Broad Compatibility and Versatile Application

The mineral components of the disclosed formulation-specifically the smectite and zeolite fractions-are subjected to particle-size engineering to achieve a controlled distribution characterized by a median particle diameter D₅₀ in the range of approximately 1-50 micrometers measured by laser diffraction per ISO 13320, and a D₉₀ not exceeding 100 micrometers. This finely micronized particle profile is critical to ensuring compatibility with a wide range of agricultural deployment methods, including both liquid and solid application systems.

The selected particle-size distribution enables:
i. Compatibility with standard fertigation systems, particularly those employing filtration screens rated at 120-150 mesh, thereby allowing for the delivery of the formulation via drip irrigation or overhead spray systems without observable agglomeration or nozzle obstruction under standard fertigation conditions.
ii. Spray-ability through conventional agricultural sprayers, including boom sprayers, backpack units, and pickup-mounted tank systems equipped with agitation mechanisms to maintain suspension uniformity.
iii. Uniform dispersion in soil when applied via broadcast spreading or mechanical incorporation methods, facilitating even distribution of mineral components throughout the target soil profile.

This particle-engineering approach enhances the versatility of the formulation, supporting a broad range of agronomic practices while maintaining the functional integrity of both the mineral and microbial components.

### 1.3 Adaptive Formulation Selection

The disclosed system employs a rule-based or AI-assisted model-driven approach to determine the optimal ratios of formulation components based on site-specific soil diagnostic data. This adaptive formulation methodology enables the tailoring of soil-conditioning blends to match the agronomic requirements and limitations of individual locations.

Key input parameters include soil texture, cation exchange capacity (CEC), electrical conductivity (EC), organic matter content (OM), and available water capacity (AWC). These variables are analyzed to define application objectives—such as improved water-use efficiency, nutrient retention, crop yield, or soil regeneration—and guide the selection of proportional concentrations of the three functional components: sodium smectite, zeolite, and AMF.

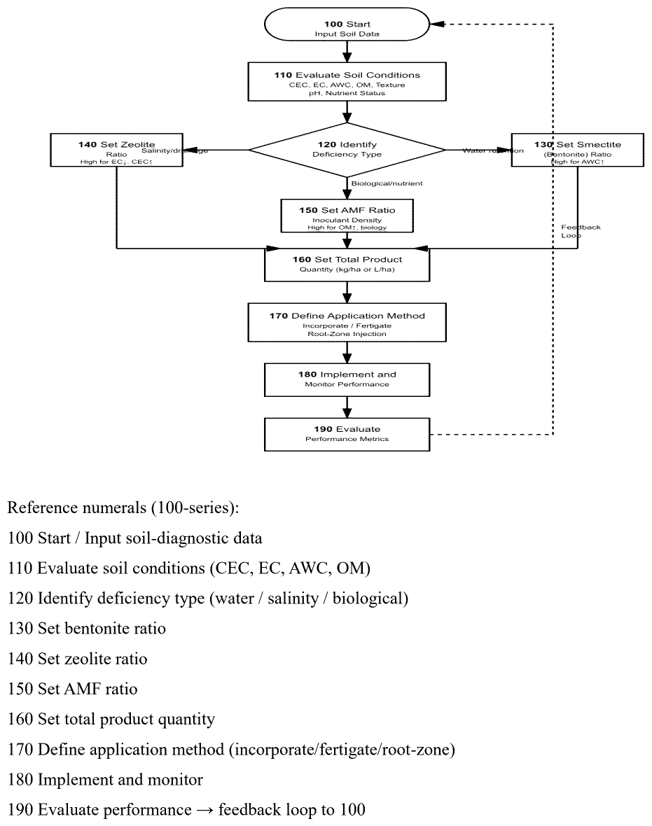

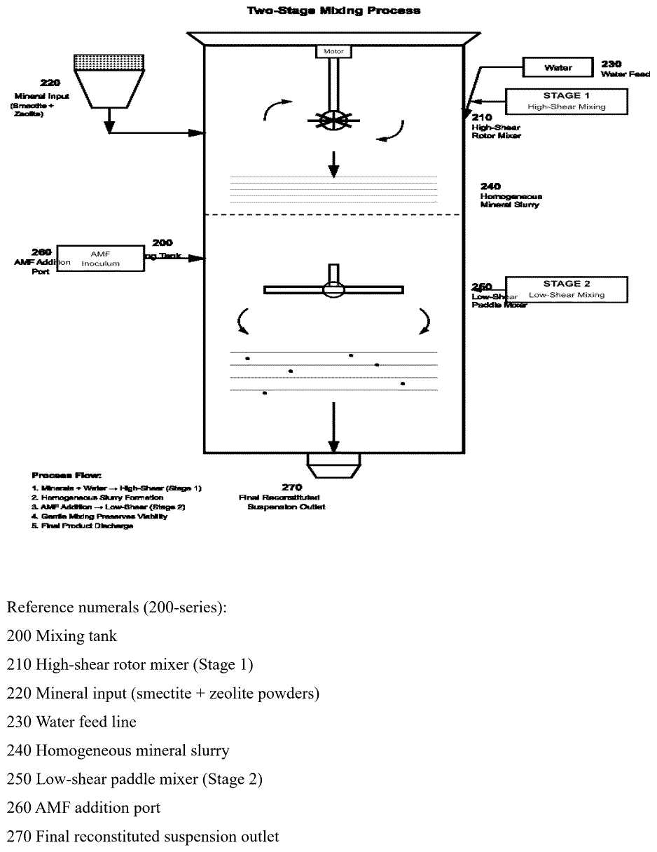

The process is illustrated in FIG. 1, which presents an adaptive formulation workflow wherein diagnostic inputs (100-110) feed into decision nodes (120-170) that define component ratios and application methods, followed by implementation and feedback (180-190) for iterative refinement.

This adaptive strategy ensures that the soil-conditioning composition is not a one-size-fits-all product, but rather a dynamic system responsive to localized soil constraints and crop management goals. Mixing of the mineral components according to the target ratio may be performed either at the production stage, or on-farm preparation facility licensed for microbial handling via dry premixing into standardized powder formulations (e.g., 30:70 by weight+AMF), or directly in the field, where proportions may be adjusted based on site-specific soil diagnostics. In all cases, the addition of water to prepare the slurry or suspension is carried out on-site, immediately prior to application.

### 1.4 Two-Stage Agronomic Protocol

The invention further provides a two-stage application protocol designed to optimize both the physical modification of the soil matrix and the biological efficacy of arbuscular mycorrhizal fungi (AMF) inoculation. This sequential methodology separates the application of the mineral and biological components to preserve microbial viability and enhance root colonization efficiency.

In the first stage, the mineral fraction—comprising micronized smectite and zeolite—is applied to the soil to improve structural properties, water retention, and nutrient buffering capacity. This conditioning phase alters the soil environment to be more conducive to microbial establishment.

In the second stage, AMF propagules are introduced using low-shear application techniques, such as root-dipping or incorporation into planting furrows, to minimize mechanical stress and maintain propagule viability. This delayed inoculation allows the fungi to colonize roots in a matrix that has been preconditioned for optimal symbiotic interaction.

By temporally separating mineral application from microbial inoculation, the protocol enhances both the abiotic and biotic contributions of the formulation, resulting in improved plant performance and soil regeneration.

This process is schematically represented in FIG. 2, illustrating the distinct high-shear and low-shear mixing stages (210, 250) and corresponding component inputs (220, 260).

### 1.5 Preservation of Biological Viability

To ensure the functional integrity of the biological component—specifically, the arbuscular mycorrhizal fungi (AMF) propagules—the formulation and handling protocols are engineered to maintain high levels of microbial viability throughout storage, transport, and application.

Key formulation parameters include a pH range of approximately 5.5 to 7.5, ionic strength not exceeding 50 millimolar, and the use of low-shear agitation, not exceeding 100 s⁻¹, during mixing and handling processes. These conditions are critical to preventing physical or chemical stress that could compromise the viability of the AMF propagules.

In addition, the system optionally employs a staged addition protocol, wherein the AMF component is introduced in a separate step—post-mineral mixing—to avoid exposure to high-shear environments or potentially destabilizing chemical conditions.

Under these conditions, AMF propagule viability of ≥80% is maintained under ambient storage and field-use conditions, ensuring effective colonization and symbiotic functionality upon application.

### 2. Composition

### 2.1 Core Components

i. Smectite Clay Fraction
   i. Particle Size: D₅₀=1-50 μm; D₉₀≤100 μm
   ii. Representative Types: Sodium bentonite, calcium bentonite, hectorite, saponite, or combinations thereof
   iii. Optional Surface Treatment: Acid activation, thermal treatment, or exfoliation to increase surface area and reactivity
   iv. Function: Provides water retention through interlayer swelling and a high specific surface area (typically 600-800 m²/g), while contributing significantly to the overall CEC of the formulation

### Particle Size Selection and Comparative Advantages:

The invention strategically employs micronized smectite in the 1-15 μm range to balance performance, regulatory compliance, safety, and manufacturing scalability. Unlike nano-scale particles, which pose potential safety and regulatory concerns, or macro-scale particles, which lack dispersion efficiency, the selected micronization provides optimal soil interaction.

TABLE 1
Comparison of Particle Size Strategies for Soil Amendment

| | Nanoscale | Micronized (10-20 μm) | Macro-scale |
|---|---|---|---|
| Property | (<2 μm) | (This Invention) | (>50 μm) |
| Surface Area | Very high (>50 m²/g) | High (~5-15 m²/g) | Low (<1 m²/g) |
| Regulatory Risk | High (classified as | None (conventional | None |
| | nanomaterial) | mineral) | |
| Inhalation Safety | Potential hazard | Safe for agricultural | Safe |
| | (respirable nanoparticles) | handling | |
| Production Cost | High (specialized | Moderate (scalable | Low (bulk milling |
| | processes) | grinding) | |
| Soil Dispersion | Excellent but unstable | Excellent and stable | Poor (uneven |
| | (prone to aggregation) | | distribution) |
| Compatibility with | Limited (non-structured | High (supports | Low (poor |
| Microbial Life | integration) | colonization) | rhizospheric contact) |
| Application | Limited (specialized | High (slurry, powder, | Limited (granular |
| Flexibility | suspensions) | granules) | only) |

ii. Zeolite Fraction
   i. Particle Size: D₅₀=1-50 μm; D₉₀≤100 μm
   ii. Representative Types: Clinoptilolite, chabazite, mordenite, synthetic zeolites (e.g., Zeolite 4A), or combinations thereof
   iii. Function: Delivers a high cation exchange capacity (typically 100-200 meq/100 g), enabling selective adsorption and slow release of essential nutrients such as ammonium (NH₄⁺) and potassium (K⁺), while contributing structural porosity to the soil matrix
iii. Arbuscular Mycorrhizal Fungi (AMF) Propagules
   i. Concentration: 10³ to 10⁷ viable propagules per gram of microbial fraction or per gram of total composition, depending on formulation
   ii. Representative Species: May include Rhizophagus irregularis (syn. Glomus intraradices), Funneliformis mosseae (syn. Glomus mosseae), Claroideoglomus etunicatum, Rhizophagus clarus, or other agriculturally beneficial AMF species, formulated as either monocultures or consortia
   iii. Biological Forms: Spores, colonized root fragments, extraradical hyphae, or combinations thereof, typically delivered in a dry or suspended medium
   iv. Function: Facilitates symbiotic root colonization to improve nutrient and water uptake—particularly phosphorus and micronutrients—while enhancing drought resilience, soil structure, and microbial diversity in the rhizosphere

TABLE 2
Core Components of the Soil Conditioning Composition

| Component | Representative Types | Primary Function |
|---|---|---|
| Smectite clay | Bentonite (montmorillonite), | Swelling clay enhancing available |
| | hectorite, saponite | water capacity (AWC) and cation |
| | | exchange capacity (CEC) |
| Zeolite | Clinoptilolite, chabazite, | Ion-exchange mineral for nutrient |
| | mordenite, synthetic 4A | retention, particularly NH₄⁺ and K⁺ |
| Component | Representative Types | Primary Function |
| AMF | Rhizophagus irregularis, | Symbiotic root colonization for |
| propagules | Funneliformis mosseae, | nutrient uptake and soil |
| | Claroideoglomus etunicatum, | biological activity |
| | Endomycorrhiza, | |
| | Glomus etunicatum, | |
| | Glomus microaggregatum, | |
| | Glomus intraradices, | |
| | Glomus claroideum, | |
| | Glomus mosseae, | |

### 2.2 Component Ratios

On a dry-weight basis, the formulation comprises the three core components-(A) sodium smectite clay, (B) zeolite, and (C) arbuscular mycorrhizal fungi (AMF) propagules-within the following preferred ratio ranges:
(A):(B):(C)=(10-90):(10-90):(0.05-10)

These ratios are adaptively selected based on diagnostic analysis of the target soil, as described in Section 1.3 (Adaptive Formulation Selection) and depicted in FIG. 1. Representative formulations include:
i. Water-Retention-Biased: (60-80):(20-35):(2-5)
   a. Optimized for sandy soils exhibiting low water-holding capacity and high infiltration rates.
ii. Nutrient-Efficiency-Biased: (30-40):(50-70):(1-3)
   a. Suitable for soils with low cation exchange capacity (CEC) and high nutrient leaching potential.
iii. Balanced: (40-50):(40-50):(1-5)
   a. Designed for loam or mixed-texture soils requiring moderate improvement across physical, chemical, and biological parameters.
iv. Regeneration-Biased: (35-45):(35-45):(5-10)
   a. Intended for severely degraded or biologically inert soils where microbial reactivation and root symbiosis are primary objectives.

These ratio sets are non-limiting examples, and other compositions may be derived through the adaptive workflow in response to specific soil diagnostics and agronomic objectives.

### 2.3 Optional Additives

To improve formulation stability, microbial viability, application performance, or agronomic efficacy wherein the composition optionally includes one or more additives selected from the group consisting of humic acid, fulvic acid, humate salts, hydrocolloid stabilizers, surfactants, dispersing agents, pH buffers, and protective agents for AMF, each present at up to 10% by weight

### Representative Optional Additives Include:

i. Humic Substances: 0.1-10 wt. % (dry basis)
   a. Types: Humic acid, fulvic acid, and humate salts (e.g., sodium, potassium, ammonium humate)
   b. Functions: Improve soil structure, increase cation exchange capacity (CEC), chelate micronutrients, and facilitate AMF establishment in the rhizosphere
ii. Hydrocolloid Stabilizers: 0.05-2.0 wt. % (based on reconstituted suspension)
   a. Types: Xanthan gum, guar gum, sodium alginate, carboxymethylcellulose
   b. Functions: Enhance suspension stability by reducing sedimentation and maintaining homogeneous dispersion of mineral particles during application
iii. Surfactants and Dispersing Agents: 0.01-0.5 wt. %
   a. Types: Lignosulfonates, alkyl polyglucosides, fatty alcohol ethoxylates
   b. Functions: Improve wettability, promote soil penetration, and enhance dispersion of mineral particles within liquid carriers or during soil application
iv. pH Buffers: As required to maintain pH within 5.5-7.5 in the reconstituted state
   a. Types: Phosphate buffers, citrate buffers, carbonate and bicarbonate systems
   b. Functions: Stabilize pH to preserve AMF viability and ensure compatibility with typical soil chemistries
v. Protective Agents for AMF: 0.5-5.0 wt. % (based on microbial fraction)
   a. Types: Trehalose, maltodextrin, polyvinylpyrrolidone (PVP), skim milk powder
   b. Functions: Provide cryoprotection and desiccation resistance, thereby maintaining AMF viability during storage and transport

These optional additives may be included individually or in combination, depending on the specific formulation requirements and delivery method selected. Their incorporation does not compromise the integrity of the core tri-component system but instead augments its performance under variable handling and environmental conditions.

### 2.4 Physical and Chemical Properties (Reconstituted Suspension)

When the soil-conditioning composition is reconstituted with water at a ratio of approximately 1:30 to 1:50 (w/w)—i.e., one-part dry composition to 30-50 parts water—the resulting suspension exhibits the following physical and chemical properties, optimized for agronomic application and biological compatibility:
i. Particle Size Distribution: ≥95% by mass passes through a 150-mesh (106 μm) screen; ≥98% passes through a 120-(125 μm) screen without observable agglomeration or nozzle obstruction under standard fertigation conditions, ensuring compatibility with standard fertigation and spray systems
ii. pH: Between 5.5 and 7.5, maintaining a biologically favorable environment for AMF viability and plant root interface compatibility
iii. Ionic Strength: Not exceeding 50 millimolar (mM) to prevent osmotic stress to microbial propagules and maintain chemical stability of the suspension
iv. Viscosity: In the range of 0.4-1.2 Pascal-seconds (Pas) at a shear rate of 10 s⁻¹, as measured by rotational viscometry, facilitating easy pumping and spray-ability
v. Zeta Potential: Between −20 and −40 millivolts (mV), indicating a stable colloidal system with minimal aggregation of suspended particles
vi. Sedimentation: No significant settling occurs during application when subjected to continuous low shear mixing, maintaining uniformity throughout the fertigation or spraying process

### Storage Properties (Dry and Concentrated Forms):

i. AMF Viability: Maintains ≥80% viability of propagules after 3-6 months under ambient storage conditions (15-30° C., 30-70% relative humidity)
ii. Moisture Content:
   Dry powder formulations: ≤10 wt. % moisture
   Concentrated paste formulations: ≤30 wt. % moisture

These characteristics ensure both the functional performance of the mineral fractions and the biological integrity of the AMF component during storage, handling, and field application.

### 3. Manufacturing Process

### 3.1 Mineral Fraction Preparation

The mineral fraction of the composition—comprising smectite and zeolite—is prepared through a controlled series of steps that ensure consistency in particle size, purity, and suitability for biological integration. The process includes material sourcing, micronization, optional sanitization, and quality control, as detailed below.

### (a) Sourcing and Quality Control of Raw Materials

i. Raw smectite clays (e.g., bentonite, hectorite) and zeolites (e.g., clinoptilolite, chabazite) are sourced from verified geological deposits.
ii. Incoming materials are subjected to quality assurance protocols to confirm:
   a. Mineralogical purity
   b. Cation exchange capacity (CEC)
   c. Particle size distribution
   d. Absence of heavy metals, pathogens, and other contaminants

### (b) Micronization Process

iii. Smectite and zeolite are separately micronized via jet milling, air classification, or wet milling to achieve a target particle-size distribution:
   a. D₅₀: 1-15 μm
   b. D₉₀: ≤40 μm
iv. Jet milling is conducted under controlled conditions (classifier speed, feed rate) to prevent thermal degradation of material properties.
v. In certain embodiments, co-micronization of smectite and zeolite is performed to enhance particle-particle association and ensure formulation homogeneity.
vi. To reduce microbial load, mineral powders may undergo thermal treatment (e.g., 60-80° C. for 2-4 hours) or ultraviolet (UV) irradiation. These sanitization steps are conducted prior to biological component incorporation to prevent AMF degradation.

### (c) Quality Control of Final Mineral Blend

The blended mineral fraction is analyzed to verify:
i. Compliance with specified component ratios (smectite to zeolite)
ii. Particle-size distribution, using laser diffraction or standard sieve analysis
iii. Absence of microbial contamination post-sanitization
iv. Reconstituted properties, including pH and electrical conductivity (EC)

### (d) Formulation and Mixing Methodology

In a preferred embodiment, the full formulation is assembled using a two-stage mixing protocol as illustrated in FIG. 2. The method separates the high-shear dispersion of mineral components from the low-shear incorporation of AMF propagules, thereby maintaining microbial viability while ensuring uniform mineral distribution.

This dual-phase mixing approach enables on-site or near-site formulation using conventional agricultural equipment (e.g., tank mixers, paddle mixers) and does not require specialized infrastructure, making it adaptable for decentralized agricultural applications.

### 3.2 Two-Stage Mixing Method

The manufacturing and application process utilizes a two-stage mixing protocol designed to optimize the dispersion of mineral components and preserve the biological integrity of arbuscular mycorrhizal fungi (AMF) propagules. This process, illustrated in FIG. 2, is compatible with standard agricultural equipment and enables on-site or near-site preparation of the final formulation.

### Stage 1: High-Shear Mineral Dispersion Phase

i. The dry, micronized mineral components—smectite and zeolite—are introduced into a mixing tank (200) containing water, where they are subjected to high-shear mixing using a rotor mixer (210).
ii. Mixing is conducted at speeds ranging from 500 to 2,500 revolutions per minute (rpm) for approximately 20 to 30 minutes, or until a homogeneous mineral slurry (240) is formed.
iii. This high-shear environment facilitates deagglomeration, enhances surface activation, and ensures uniform dispersion of the mineral particles in the aqueous phase.
iv. Notably, the method does not require nanoscale exfoliation, thereby avoiding regulatory and safety issues associated with nanomaterials while still achieving sufficient surface area and suspension stability for field use.

### Stage 2: Low-Shear Biological Incorporation Phase

i. Upon completion of the mineral dispersion, AMF propagules or other biological agents are added via a designated inoculation port (260) to the homogeneous slurry.
ii. Mixing continues using a low-shear paddle or anchor mixer (250) operated at less than 300 rpm, sufficient to maintain uniform distribution without compromising microbial viability.
iii. This phase continues throughout the application window, ensuring that biological components remain suspended and viable during fertigation, spraying, or direct soil incorporation.
iv. The low-shear environment is critical to prevent mechanical damage to AMF structures (e.g., spores, hyphae) and to preserve colonization potential upon soil contact.

The final reconstituted suspension (270) is immediately suitable for field application and maintains both mineral homogeneity and biological functionality. The entire two-stage process is compatible with widely available agricultural equipment such as slurry tanks, sprayer systems, and irrigation feed tanks, facilitating decentralized, scalable deployment. Where such equipment is not readily available, the system can be fabricated locally using standard components, enabling flexible implementation in resource-constrained or remote settings.

### 3.3 Product Formats

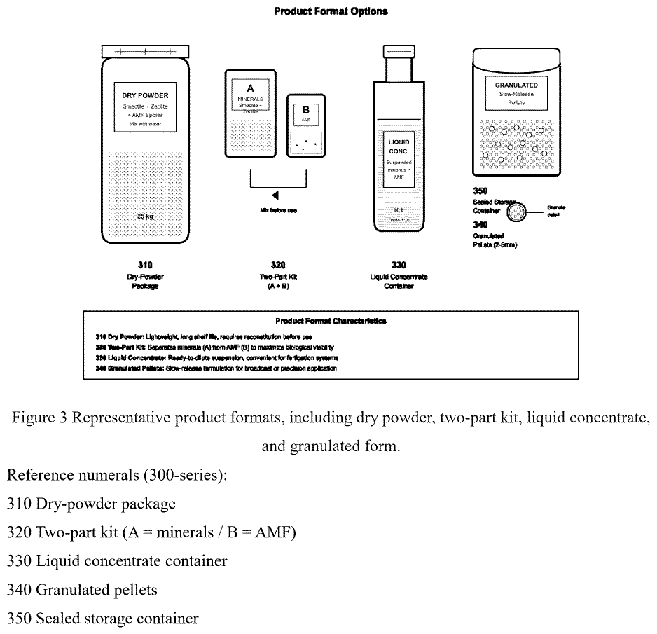

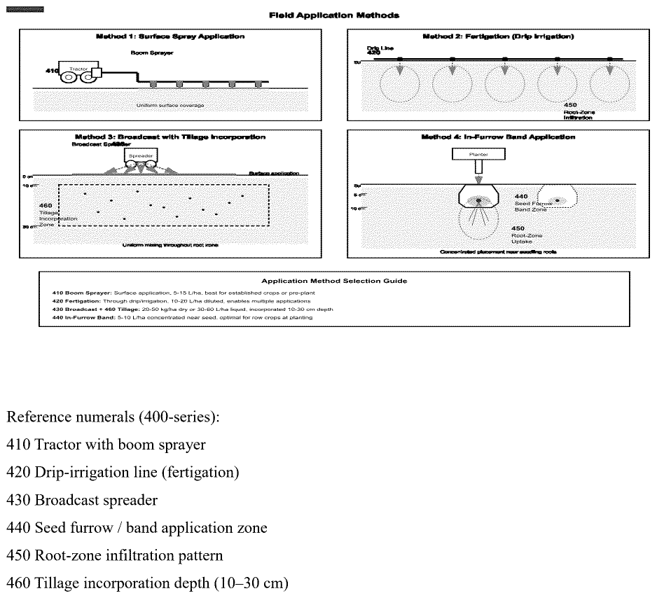

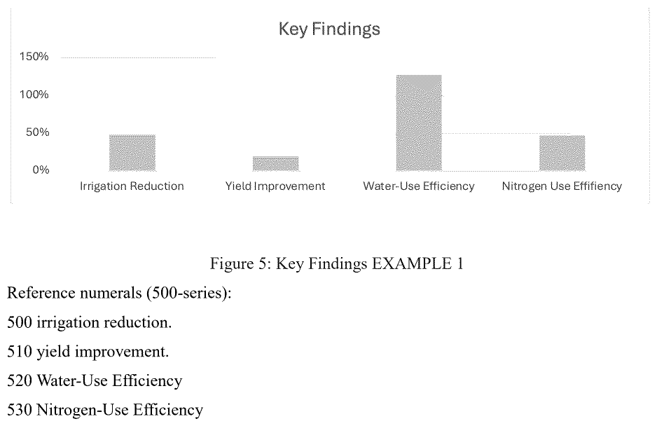

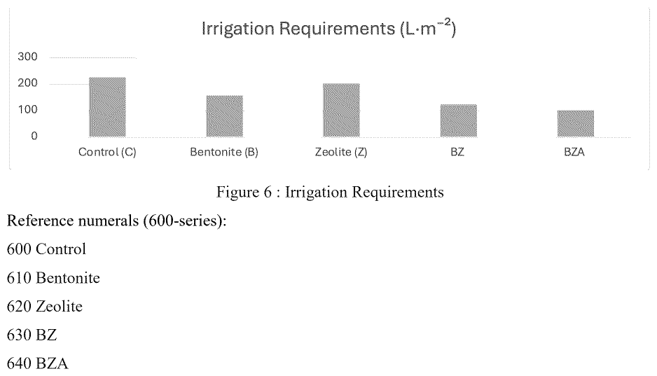

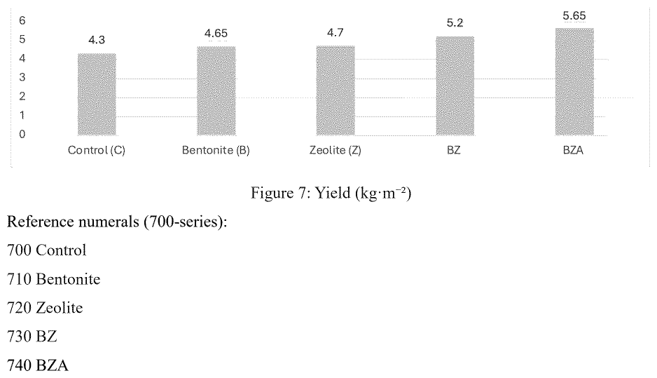

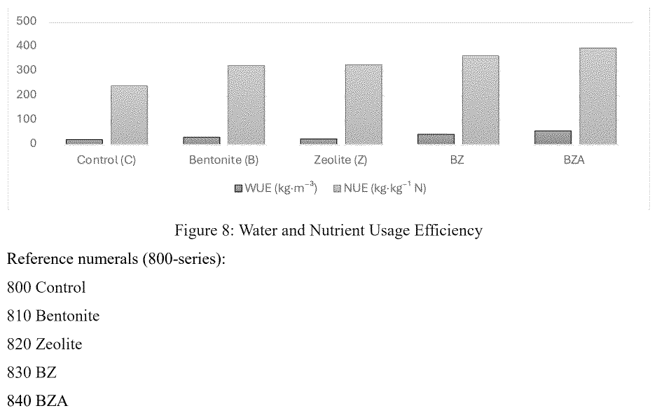

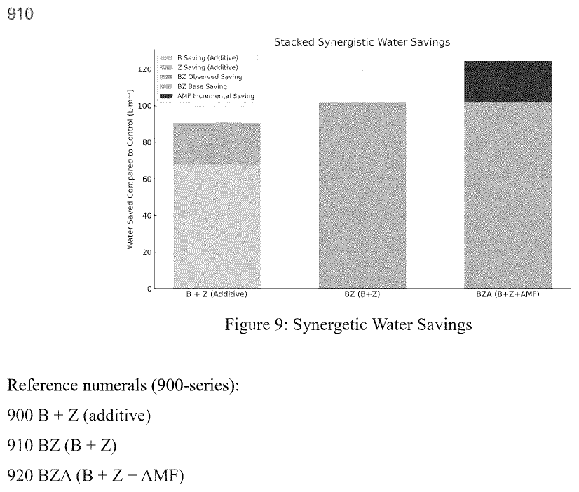

The disclosed soil-conditioning composition may be packaged and distributed in a range of physical formats to accommodate diverse application methods, storage requirements, and operational contexts. These product variants, depicted in FIG. 3, include a pre-mixed dry powder, a two-part kit, and liquid concentrate. Each format offers distinct advantages in terms of shelf life, ease of use, and compatibility with existing agricultural infrastructure.

### (1) Dry Powder Formulation (310)

i. The composition may be provided as a pre-blended dry powder containing micronized smectite clay, zeolite, and AMF propagules in predetermined ratios. Alternatively, the components may be supplied separately—either as bentonite alone, zeolite alone, or as a two-part mineral blend and biological inoculum—to allow on-site mixing and formulation adjustment based on soil-specific requirements.
ii. Moisture content is maintained at ≤10% by weight, and the product is sealed in moisture-impermeable containers to prevent degradation during storage.
iii. This format supports single-stage preparation by reconstituting the powder with water followed by low-shear mixing for approximately 30 minutes prior to field application.
iv. While operationally convenient, the co-formulation of AMF in this format limits long-term microbial viability.
v. Estimated shelf life: 6 to 9 months under ambient storage conditions (15-30° C., 30-70% RH).

### (2) Two-Part Kit Format (320)

i. The composition may alternatively be divided into two physically and functionally distinct components to preserve AMF viability and extend shelf life:
   Part A: a dry mineral blend containing micronized smectite and zeolite.
   Part B: a separate biological fraction containing AMF propagules, either in dry, suspended, or encapsulated form.
ii. Parts A and B are packaged independently, preventing premature biological degradation or physical incompatibility during storage.
iii. The user conducts sequential mixing at the point of use, beginning with high-shear dispersion of Part A into water to create a homogeneous slurry, followed by low-shear incorporation of Part B to preserve microbial structure and viability.
iv. The two-part system is ideal for on-demand formulation, scalable deployment, and field mixing using standard agricultural tanks, sprayers, or irrigation systems.
v. Estimated shelf life:
   Part A: 12 to 18 months
   Part B: 9 to 12 months, depending on carrier medium and storage conditions.

### (3) Liquid Concentrate (330)

i. A ready-to-use aqueous suspension containing micronized mineral components and AMF may be formulated with adjusted pH (5.5-7.5) and ionic strength (≤50 mM) to maintain microbial viability.
ii. Requires intermittent agitation or recirculation to prevent sedimentation and maintain uniformity during storage and application.
iii. Designed for direct use in fertigation systems, overhead irrigation, or mechanized spraying.
iv. Estimated shelf life: 3 to 6 months when stored in cool, dark conditions.

Each format may be selected based on regional logistics, equipment availability, and agronomic objectives, ensuring the formulation's adaptability across use cases.

### 4. Adaptive Formulation Selection

A central innovation of the disclosed invention lies in its adaptive formulation strategy, which selects component ratios and application rates based on quantitative soil diagnostic data. This allows the composition to be customized to site-specific soil limitations, improving both agronomic efficiency and environmental sustainability.

The adaptive process is represented schematically in FIG. 1, where soil data inputs (100) are evaluated (110), followed by formulation logic (120-170), implementation (180), and performance feedback (190) to enable iterative optimization.

### 4.1 Soil Diagnostic Parameters

Soil parameters are measured using standard agronomic methods, including:
i. Texture (sand, silt, clay %): Measured using the USDA hydrometer method or equivalent
ii. Cation Exchange Capacity (CEC): Determined using IN ammonium acetate at pH 7.0, reported in cmol(+)/kg
iii. Electrical Conductivity (EC): Measured from saturated paste extract, expressed in dS/m or mS/cm
iv. Organic Matter (OM): Assessed via loss-on-ignition or Walkley-Black method, in wt. %
v. Available Water Capacity (AWC): Derived using the pressure plate method at −33 kPa and −1500 kPa, in m³/m³
vi. Soil pH: Measured using 1:1 soil: water or 0.01M CaCl₂) solution

Optional diagnostics may include nutrient levels (N, P, K), infiltration rate, bulk density, and cropping history, which can further refine formulation decisions.

### 4.2 Decision Rules for Ratio Selection

The adaptive formulation engine applies a rule-based logic or machine learning model trained on soil-crop-performance data. The system maps specific soil conditions to target formulation ratios and co-formulants. Representative logic rules include:

### Rule 1: High Sand Content or Low Water-Holding Capacity

i. Trigger: Sand ≥70% OR AWC≤0.08 m³/m³
ii. Action: Increase smectite fraction; maintain moderate zeolite; increase AMF
iii. Recommended Ratio (A:B:C): (60-80):(20-35):(3-5)
iv. Rationale: Smectite improves water retention; AMF enhances root water uptake
   Rule 2: Low CEC with Nutrient Leaching Risk
i. Trigger: CEC <10 cmol(+)/kg AND high sand or rainfall
ii. Action: Increase zeolite; maintain moderate smectite; increase AMF
iii. Recommended Ratio (A:B:C): (30-40):(50-70):(2-4)
iv. Rationale: Zeolite retains nutrients via ion exchange; AMF increases nutrient use efficiency

### Rule 3: High Salinity

i. Trigger: EC >4 dS/m (saturated paste extract)
ii. Action: Moderate total dose; co-formulate with humic acids; increase AMF
iii. Recommended Ratio (A:B:C): (35-45):(35-45):(3-6)
iv. Co-formulant: Add 2-5 wt. % humic or fulvic acid
v. Rationale: Balances sodium, improves osmotic tolerance, supports AMF resilience

### Rule 4: Low Organic Matter

i. Trigger: OM<1 wt. %
ii. Action: Increase AMF dose; add humic substances
iii. Recommended Ratio (A:B:C): (35-45):(35-45):(5-10)
iv. Co-formulant: Add 0.5-5 wt. % humic/fulvic acid
v. Rationale: AMF and humates stimulate microbial activity and organic accumulation

### Rule 5: Balanced Loam Soil

i. Trigger: Sand 40-60%, silt 20-40%, clay 10-25%; CEC 10-20 cmol(+)/kg; AWC 0.10-0.15 m³/m³
ii. Action: Apply balanced tri-component formulation
iii. Recommended Ratio (A:B:C): (40-50):(40-50):(1-3)
iv. Rationale: Maintenance dose for soils with moderate constraints

TABLE 3
Adaptive Formulation Selection

| | | Recommended | Co-Formulants | |
|---|---|---|---|---|
| Rule | Trigger | Ratio (A:B:C) | (Optional) | Rationale |
| 1: High | Sand ≥70% OR | (60-80):(20- | | Smectite improves water |
| sand/low | AWC ≤0.08 | 35):(3-5) | | retention; AMF enhances |
| AWC | m³/m³ | | | uptake |
| 2: Low | CEC <10 | (30-40):(50- | — | Zeolite retains nutrients; |
| CEC with | cmol(+)/kg + | 70):(2-4) | | AMF boosts efficiency |
| leaching | sandy/high | | | |
| | rainfall | | | |
| 3: High | EC >4 dS/m | (35-45):(35- | 2-5 wt. % | Humates and AMF |
| salinity | | 45):(3-6) | humic/fulvic | improve tolerance to salt |
| | | | acid | stress |
| 4: Low | OM <1 wt. % | (35-45):(35- | 0.5-5 wt. % | Stimulates microbial |
| OM | | 45):(5-10) | humic/fulvic | activity and OM |
| | | | acid | accumulation |
| 5: | Moderate | (40-50):(40- | — | General soil maintenance |
| Balanced | texture, CEC, | 50):(1-3) | | |
| loam | AWC | | | |

### 4.3 Application Rate Selection

In addition to selecting component ratios, the total application rate (by area) is adjusted based on soil texture, fertility status, and project goals (e.g., maintenance vs. regeneration):

### Mineral Fractions (A+B)

i. General Range: 100-10,000 kg·ha⁻¹
ii. Typical Operational Range: 200-1,500 kg·ha⁻¹
iii. High Rates: Used for coarse-textured soils, severely degraded areas, or soil regeneration projects
iv. Low Rates: Applied to loam or silt soils with moderate CEC and AWC

### AMF Fraction (C)

i. Range: 0.5-5.0 kg ha-1 (active propagule mass)
ii. High Rates: Recommended for biologically poor soils, perennial crops, or ecological restoration
iii. Low Rates: Suitable for annual row crops or soils with moderate biological function

This flexible dosing model ensures that both physical and biological inputs are calibrated to local conditions, supporting more effective and sustainable soil management.

### 5. Application Methods and Agronomic Protocol (400-460)

The disclosed composition is compatible with a wide range of standard agricultural equipment and may be applied through multiple agronomic protocols depending on crop type, field scale, equipment availability, and grower preference. Application methods are designed to ensure effective delivery of both mineral and biological components, while maintaining microbial viability and achieving optimal root-zone integration.

### 5.1 Primary Application Methods

The four principal application methods include banded/in-furrow application, spray application, fertigation, and broadcast with mechanical incorporation. Each method offers specific agronomic advantages, as detailed below and illustrated in FIG. 4.

### (1) Banded/In-Furrow Application (440)—(Preferred and Optimal Method for Row Crops and Transplants)

i. Procedure:
   Reconstitute composition in water at a 1:30 to 1:50 (w/w) ratio.
   Apply as a liquid band along the planting row or directly into the seed furrow using a liquid applicator.
   Plant seeds or transplants into the treated zone.
   Apply 8-25 m³·ha⁻¹ of water as the carrier volume.
ii. Advantages:
   Concentrates active components in the root zone
   Reduces total product required per hectare
   Highly effective for vegetables, maize, cotton, and transplanted crops

### (2) Spray Application (410)—(Efficient, Flexible, and Equipment-Compatible)

i. Procedure:
   Reconstitute at 1:30 to 1:50 (w/w) with water in a spray tank.
   Apply via:
      Tractor-mounted boom sprayer
      Backpack sprayer
      Pickup- or ATV-mounted sprayer
   Apply to soil surface pre-plant or between crop rows.
   Lightly incorporate or irrigate post-application.
ii. Spray Volume: 40-70 m³·ha⁻¹
iii. Advantages:
   Rapid application
   Minimal soil disturbance (ideal for no-till or reduced-till systems)
   Effective in inter-row treatments
   Compatible with portable or small-farm equipment

### (3) Fertigation (420)—(Preferred for Irrigated High-Value Crops)

i. Procedure:
   Reconstitute at 1:30 to 1:50 (w/w) in a fertigation tank.
   Inject through drip, micro-sprinkler, or sprinkler systems using:
      Venturi injector
      Positive-displacement pump
      Pressure differential tank
   Compatible with 80-200 mesh filters (most commercial systems).
   Flush system with clean water post-application.
ii. Advantages:
   Utilizes existing irrigation infrastructure
   Enables in-season application
   Reduces labor input
   Suitable for vineyards, orchards, greenhouses, and intensive horticulture
iii. Note: While fertigation is logistically efficient, spray or in-furrow application provides superior soil-conditioning effects due to greater mineral-soil contact.

### (4) Broadcast+Mechanical Incorporation (430, 460)—(High-Capacity Method for Field-Scale Implementation)

i. Procedure:
   Apply as dry powder using a broadcast spreader or as a reconstituted suspension using spray equipment.
   Incorporate into the soil to a depth of 10-30 cm using tillage equipment such as:
      Disc harrow
      Rotovator
      Chisel plow
ii. Timing:
   Typically applied pre-plant, allowing 1-7 days for soil equilibration.
iii. Application Rate:
   Mineral (A+B): 1,000-2,000 kg·ha⁻¹
   AMF (C): 0.5-3.0 kg·ha⁻¹
iv. Advantages:
   Full integration of product into root zone
   No filter systems required
   Scalable to large acreages

### 5.2 Two-Stage Agronomic Protocol (Preferred for Transplanted Crops)

For transplanted crops such as vegetables, tree seedlings, ornamentals, a two-stage protocol ensures both optimal soil conditioning and effective AMF root colonization.

### Stage 1: Soil Conditioning

i. Timing: 3-7 days prior to transplanting (range: 1-30 days)
ii. Composition: Mineral fraction only (A+B), or full composition with reduced AMF load
iii. Application: Via spray application or fertigation, followed by light irrigation
iv. Objective: Improve soil structure (AWC, CEC), reduce compaction, and enhance biological receptivity

### Stage 2: Root Inoculation

i. Timing: Immediately prior to transplanting (0-5 hours)
ii. Composition: AMF propagule suspension (component C), optionally with 0.5-2.0 wt. % humic or fulvic acid
iii. Application: Root-dipping transplants in AMF suspension for 2-10 minutes
iv. Suspension Concentration: 10⁴-106 propagules per liter
v. Objective: Ensure direct contact of AMF with plant roots to maximize early symbiosis and improve survival in the field
vi. Rationale: Temporal separation of mineral and microbial stages reduces osmotic stress and creates a conditioned soil matrix conducive to AMF colonization. A delay of 3-7 days between stages is optimal.

### Transplanting and Optional Follow-Up

i. Transplant treated seedlings into conditioned beds or rows.
ii. Resume standard irrigation scheduling.
iii. Optionally, apply a follow-up fertigation at 10-20% of the initial dose 2-4 weeks post-transplant to reinforce AMF colonization.

### 5.3 Compatibility Notes

The disclosed composition has been specifically engineered for compatibility with a broad range of conventional agricultural infrastructure, particularly irrigation and application systems commonly found on farms of varying scale. The following considerations clarify the scope and performance of the formulation in this context:
i. Compatibility with Standard Mesh Screens:
   a. Most commercial fertigation systems utilize inline filtration screens rated between 80 and 200 mesh to prevent emitter clogging. The micronized particle-size distribution of the composition (≥95%<106 μm) ensures unobstructed passage through these screens under normal operating conditions. While this compatibility enables seamless fertigation integration, it is considered a secondary convenience feature—not the primary technical objective of the invention.
ii. Efficacy of Fertigation in Specific Contexts: In certain embodiments, fertigation may offer superior delivery performance compared to traditional surface or subsurface methods such as broadcasting, spray-and-incorporation, or in-furrow banding. This is particularly relevant in:
   a. Coarse-textured soils, where rapid infiltration supports deeper penetration of microbial and mineral components
   b. Arid or semi-arid climates, where surface-applied formulations may desiccate before incorporation
   c. Systems requiring uniform product distribution over large or irregular plots

Fertigation promotes improved rhizosphere access, enhances AMF propagule viability, and reduces manual labor requirements.
iii. No Specialized Filtration Required:
   a. The formulation is fully compatible with existing irrigation equipment and does not necessitate any modification or addition of specialized filters, pumps, or mixing hardware. This ensures low barriers to adoption and broad operational scalability across diverse farming systems.

### 6. Mechanisms of Action

The disclosed tri-technology soil-conditioning composition acts through a synergistic integration of physical, chemical, and biological mechanisms. Each of the three core components—smectite clay, zeolite, and arbuscular mycorrhizal fungi (AMF)—addresses distinct but interrelated limitations in soil function, enabling comprehensive improvement in soil performance, plant health, and water/nutrient efficiency.

### 6.1 Smectite: Water Retention and Irrigation Reduction

### Mechanism of Action

The smectite fraction (e.g., sodium or calcium bentonite) improves soil hydrodynamics through its high surface area and swelling behavior:
i. Layered Mineral Structure: Smectite clays possess a 2:1 phyllosilicate structure, with hydrated interlayer cations (Na⁺, Ca²⁺) that promote interlayer swelling upon contact with water.
ii. Hydration and Swelling: Water molecules infiltrate the interlayers, causing clay particles to expand and disaggregate, increasing micro-porosity and enhancing the soil's ability to retain moisture.
iii. Specific Surface Area: Smectite clays exhibit a specific surface area of 600-800 m²/g, providing abundant adsorption sites for water molecules and enabling capillary retention at a range of matric potentials.
iv. Functional Effect: These physical properties expand the available water capacity (AWC) of soils by increasing the fraction of moisture held at tensions accessible to plant roots.

### Measured Agronomic Outcomes

The application of the smectite component has been observed to yield the following quantifiable benefits:
i. Increase in Soil AWC: +10% to +40% (on a volumetric basis), depending on initial soil texture and smectite application rate
ii. Reduction in Seasonal Irrigation Volume:
   10-60% reduction in sandy soils
   10-40% reduction in loam soils
iii. Extension of Irrigation Intervals:
   1.5× to 3× longer intervals between required irrigation events

These effects translate into enhanced drought resilience, reduced irrigation cost, and greater scheduling flexibility, especially under deficit irrigation regimes or in water-scarce environments.

### 6.2 Zeolite: Nutrient Retention and Leaching Reduction

### Mechanism of Action

The zeolite component—primarily clinoptilolite—functions as a cation-exchange and nutrient-buffering mineral through the following mechanisms:
i. Three-Dimensional Framework Structure: Zeolites possess a porous crystalline structure with interconnected cages and channels capable of selective cation exchange, particularly for NH₄⁺, K⁺, Ca²⁺, and Mg²⁺.
ii. Ammonium Affinity and Nutrient Retention: Clinoptilolite exhibits a high selectivity for ammonium (NH₄⁺), reducing losses of nitrogen from urea or ammonium-based fertilizers through immobilization and gradual release.
iii. Cation Exchange Capacity (CEC): The zeolite fraction can increase soil CEC by 2-10 cmol(+)/kg, depending on application rate and native soil characteristics, thereby enhancing the soil's nutrient-retention profile.
iv. Salt Mitigation: In saline or sodic soils, zeolite contributes to ionic balance by exchanging sodium ions (Na⁺) for divalent cations, reducing sodium toxicity and improving soil structure.

### Measured Agronomic Outcomes:

Nitrogen Fertilizer Reduction: 10-30% reduction in required nitrogen input, without yield loss
Improved Nutrient Retention: Decreased leaching of potassium and micronutrients
Prolonged Fertilizer Effectiveness: Nutrient availability sustained over time, aligning with plant uptake demands

### 6.3 AMF: Root Symbiosis and Water/Nutrient Access

### Mechanism of Action

Arbuscular mycorrhizal fungi (AMF) establish obligate symbiotic relationships with host plant roots and significantly enhance nutrient and water acquisition through several pathways:
i. Root Colonization and Hyphal Extension: AMF form arbuscules within root cortical cells for nutrient exchange and extend extraradical hyphae up to 100-1,000 times beyond root hairs, enabling exploration of inaccessible micropores and soil volumes.
ii. Phosphorus Uptake: AMF substantially enhance phosphorus acquisition, a key immobile nutrient in most soils, through direct scavenging and translocation to the host plant.
iii. Stress Tolerance Enhancements: Colonized plants demonstrate improved resilience to drought and salinity through:
   Hydraulic redistribution
   Osmotic regulation
   Ion exclusion and detoxification
   Enhanced root system development

### Measured Agronomic Outcomes:

i. Root Colonization: 10-60% within 30-90 days, depending on crop, species, and soil
ii. Phosphorus Uptake Increase: 10-80%
iii. Nitrogen Uptake Efficiency: 10-30% improvement
iv. Drought Tolerance: Plant survival extended by 1-3 weeks under water-limited conditions
v. Salinity Tolerance: 10-30% improvement in plant performance under saline conditions

### 6.4 Soil Biological Function and Regeneration

### AMF-Driven Soil Health Improvements

In addition to plant-specific benefits, AMF play a pivotal role in soil ecosystem function and regeneration, particularly in degraded or biologically depleted environments:
i. Glomalin Secretion and Aggregate Formation: AMF hyphae secrete glomalin, a glycoprotein that enhances soil aggregate stability by binding fine particles, promoting improved soil structure, infiltration, and aeration.
ii. Stimulation of Rhizosphere Microbiome: AMF colonization is associated with increased bacterial and fungal diversity, enriching the soil microbiome and enhancing biogeochemical cycling.
iii. Organic Matter Accumulation: Repeated AMF colonization contributes to long-term increases in soil organic matter (SOM) and overall soil fertility, particularly when paired with regenerative crop rotations or organic inputs.

### Measured Regenerative Outcomes:

Microbial Biomass Increase: 10-50% after one season
Soil Aggregate Stability: 10-30% improvement
Soil Organic Matter Accumulation: 0.1-0.5% absolute increase over 2-5 years
Site Reclamation: Successful reactivation of marginal or degraded lands for productive use

### 7. Storage, Handling, and Safety

The disclosed composition is composed entirely of naturally occurring mineral and microbial constituents and is classified as a soil conditioner rather than a pesticide or fertilizer. It is non-toxic under normal use conditions and compatible with organic production systems, subject to approval by local certifying bodies.

### 7.1 Storage Conditions

### Dry Powder Formulations:

i. Store in sealed, moisture-barrier containers (e.g., multi-layer bags or polymer-lined drums)
ii. Temperature range: 15-30° C.
iii. Relative humidity: 30-70%
iv. Avoid:
   Freezing temperatures
   Prolonged exposure to high humidity, which may degrade AMF viability or cause mineral clumping
v. Shelf life: 6-12 months under recommended conditions
   AMF viability: ≥80% for 6-9 months

### Concentrated Suspensions:

i. Store in opaque, tightly sealed containers
ii. Agitate before use to ensure uniformity
iii. Temperature range: 10-25° C.
iv. Avoid freezing
v. Shelf life: 3-6 months, depending on microbial formulation
   Viability testing recommended before application in long-term storage scenarios

### Reconstituted Suspensions (Ready-to-Use):

i. Use within 5 hours of preparation
ii. Store in a cool, shaded location
iii. Maintain continuous low-shear agitation to prevent sedimentation
iv. Do not freeze or expose to direct sunlight

### 7.2 Handling

i. Personal Protective Equipment (PPE):
   When handling dry powders, use dust masks, gloves, and eye protection to minimize inhalation and ocular exposure to fine mineral particles
   Avoid skin contact with concentrated suspensions; rinse immediately with water if contact occurs
ii. The composition is non-toxic, non-hazardous, and free of synthetic additives under standard use conditions

### 7.3 Safety and Regulatory Classification

i. Not a Pesticide:
   The composition does not contain active ingredients intended to repel, control, or mitigate pests, and does not fall under EPA pesticide classification
ii. Not a Fertilizer:
   It does not supply primary macronutrients (N, P, K) in plant-available forms at rates that would classify it as a commercial fertilizer
iii. Soil Conditioner/Amendment:
   Functions by modifying soil physical and chemical properties, particularly water retention, cation exchange capacity, and biological activity
   May be subject to state or regional soil amendment regulations, depending on jurisdiction
iv. Organic Compatibility:
   All primary ingredients—bentonite, zeolite, and AMF—are naturally occurring and generally permitted in organic production systems
   Final formulations may be eligible for OMRI listing or local organic certification, depending on co-formulants used (e.g., humic substances, stabilizers)

### Regulatory and Labeling Guidance:

i. Product labeling should emphasize:
   “Soil conditioning”
   “Water retention enhancement”
   “Nutrient retention”
   “Improved soil structure and biological function”
ii. Avoid marketing claims that imply:
   Pest control (unless separately registered)
   Fertilization (unless supplemented with approved nutrient ingredients)
   Plant growth regulation
iii. Claims related to performance (e.g., irrigation reduction, fertilizer reduction, yield increases) should be substantiated by field trials and may require data disclosure or regulatory review depending on local labeling laws.

### Terminology and Definitions

As used herein, the following terms have the meanings set forth below:
“Smectite clay”, as used in this disclosure, refers to swelling 2:1 phyllosilicate mineral that possess interlayer hydration capacity, high cation exchange potential, and expansive surface area. The term includes, but is not limited to, montmorillonite-rich bentonite, hectorite, and saponite. In the context of the present invention, the smectite clay is preferably natural sodium bentonite or calcium bentonite that has been sodium-activated, owing to their superior swelling behavior upon hydration. This swelling capacity is critical to the soil-conditioning function described herein, enabling increased porosity, water retention, and enhanced interaction with biological and mineral components.
“Zeolite” refers to microporous aluminosilicate minerals, either natural or synthetic, including but not limited to clinoptilolite, chabazite, mordenite, and synthetic zeolites such as Zeolite 4A. Zeolites are characterized by high cation exchange capacity, selective ion exchange, and structural porosity.
“Arbuscular mycorrhizal fungi (AMF)” refers to fungi of the phylum Glomeromycota that form symbiotic associations with plant roots, including genera such as Glomus, Rhizophagus, Funneliformis, Claroideoglomus, etunicatum, microaggregatum, intraradices, claroideum, mosseae, and Endomycorrhiza.
“AMF propagules” refers to viable AMF reproductive and colonization structures including spores, colonized root fragments, vesicles, extraradical hyphae, and any other structures capable of establishing mycorrhizal colonization in plant roots.
“AMF propagule quantification” is performed according to ISO 14238 Most Probable Number (MPN) method or equivalent serial dilution bioassay, with minimum detection limit of 10² propagules per gram.
“AMF viability” is assessed using vital staining methods such as INT (2-(4-iodophenyl)-3-(4-nitrophenyl)-5-phenyl-2H-tetrazolium chloride) staining, with ≥80% stained propagules considered viable. Alternative viability assays include FDA (fluorescein diacetate) staining or germination assays.
“BZ” refers to a mineral-based soil amendment comprising a physical mixture of micronized sodium smectite clay (preferably montmorillonite) and micronized zeolite (preferably clinoptilolite), wherein both components possess a median particle size (D₅₀) of less than 50 microns. The composition is formulated to enhance soil water retention, cation exchange capacity, and nutrient-holding properties through combined swelling and adsorption mechanisms.
“BZA” refers to a composite soil amendment comprising: (i) a mineral fraction composed of micronized sodium smectite clay (preferably montmorillonite) and micronized zeolite (preferably clinoptilolite), both having a median particle size (D₅₀) of less than 50 microns; and (ii) a biological inoculum comprising viable arbuscular mycorrhizal fungi (AMF) propagules, such as spores, colonized root fragments, or hyphal networks. The formulation is designed to synergistically enhance soil water retention, nutrient buffering, and plant-root symbiosis, combining the physicochemical properties of the mineral matrix with the biological functionality of mycorrhizal colonization.
“Micronized” refers to particle size reduction, typically via jet milling, air classification, or wet milling, to achieve a target particle size distribution characterized by D₅₀ (median diameter) and D90 (90th percentile diameter).
“D50” is the median particle diameter, meaning 50% by mass of particles are smaller than this value.
“D90” is the 90th percentile particle diameter, meaning 90% by mass of particles are smaller than this value.
“Reconstitutable” means capable of being converted from a dry powder, concentrate, or granulated form into a stable aqueous suspension suitable for application, using water and low- to moderate-shear mixing.
“Mesh screen” refers to a filtration screen characterized by the number of openings per linear inch. A 150-mesh screen has openings of approximately 106 μm; a 120-mesh screen has openings of approximately 125 μm. Most commercial agricultural irrigation systems include mesh filtration in the range of 80-200 mesh.
“Soil conditioning” refers to physical, chemical, or biological modification of soil properties including, but not limited to, water-holding capacity, cation exchange capacity, soil structure, porosity, and microbial activity, for the purpose of improving agricultural productivity or enabling cultivation of previously marginal sites.
“Cation exchange capacity (CEC)” is determined by standard methods such as the IN ammonium acetate method at pH 7.0 (e.g., USDA NRCS method or equivalent), expressed in units of centimoles of positive charge per kilogram of soil (cmol(+)/kg) or milliequivalents per 100 grams (meq/100 g).
“Available water capacity (AWC)” is measured via pressure plate apparatus as the difference in volumetric water content between field capacity (typically −33 kPa or −0.33 bar) and permanent wilting point (typically −1500 kPa or −15 bar), expressed in units of m³/m³ or volume percent.
“Ambient storage conditions” comprise temperature of 15-30° C. and relative humidity of 30-70%.
“Low-shear agitation” refers to mixing or agitation conditions producing shear rates ≤100 s⁻¹, suitable for maintaining AMF propagule viability during processing and reconstitution.

### 8. Experimental Data and Validation

The following examples illustrate specific embodiments of the disclosed soil-conditioning composition and methods, demonstrating the performance improvements achievable through the synergistic integration of micronized smectite clay, zeolite, and arbuscular mycorrhizal fungi (AMF). These examples are provided for illustrative purposes and are not intended to limit the scope of the invention.

### Example 1: Field Evaluation of Micronized Bentonite-Zeolite Composition Without AMF (2024)

### Objective

To evaluate the effects of a micronized bentonite-zeolite composition on irrigation requirements, fertilizer efficiency, and tomato yield under temperate garden conditions, and to compare performance against untreated control soil. All agronomic parameters were measured under identical climatic and irrigation control conditions to ensure comparability.

### Experimental Design

Location: Toronto, Ontario, Canada (43.7°N, 79.4° W)
Soil Type: Native garden loam, pH 6.4
Test Crop: Tomato (Solanum lycopersicum cv. ‘Roma’)
Total Area: 10 m² divided into four randomized blocks
Plot Size: 0.5 m² per plot
Planting Density: Three plants per plot
Growing Season: May-September 2024

### Treatment Groups:

Treatment (n=16 plots): Micronized bentonite-zeolite composition applied at 0.10 kg·m⁻²
Control (n=4 plots): No soil amendment

### Composition Specifications:

The formulation comprised:
70 wt. % micronized sodium bentonite having D₅₀=12 μm, D₉₀=35 μm. Particle size verified by laser diffraction per ASTM C1070 standard.
30 wt. % micronized clinoptilolite zeolite having D₅₀=15 μm, D₉₀=38 μm
No AMF component included in this trial

The mineral components exhibited specific surface areas of 720 m²/g (bentonite) and 45 m²/g (zeolite), with bentonite swelling capacity measured at 12.5 mL/2 g.

### Application Protocol

### Initial Application:

The composition powder was reconstituted on-site at a 1:40 (w/w) powder-to-water ratio (2.439 wt. % solids; approximately 25 g·L⁻¹) using high-shear mechanical mixing for 30 minutes to achieve complete dispersion and prevent aggregation.

The resulting suspension was applied manually to test plots at the target rate of 0.10 kg·m⁻² (equivalent to 4.1 L·m⁻² of reconstituted suspension). Control plots received an equal volume (4.1 L·m⁻²) of plain water to maintain identical initial wetting conditions.

### Subsequent Irrigation:

All plots were irrigated via drip irrigation (2 L·h⁻¹ emitters at 30 cm spacing) regulated by soil tensiometers maintaining approximately −25 kPa during vegetative growth and −20 kPa during fruiting stages, following established protocols for tomato cultivation on coarse soils.

A 120-150 mesh inline filter was installed to prevent emitter clogging, confirming that the micronized particle size distribution (≥95%<106 μm) enabled unobstructed passage through standard fertigation equipment.

Irrigation volumes were recorded using flowmeters at the block level.

### Fertilization Protocol:

To evaluate nutrient-use efficiency under realistic conditions, nitrogen fertilization was differentiated between treatments:
Control plots: 18.3 g·m⁻² (100% of soil-test recommendation)
Composition powder plots: 14.9 g·m⁻² (approximately 80% of recommendation)

Nitrogen was supplied weekly through fertigation using a balanced water-soluble fertilizer (18-18-21 NPK formulation).

### Measurements

Irrigation volume (L·m⁻²): Cumulative seasonal total per plot
Nitrogen applied (g·m⁻²): Cumulative fertigation input per plot
Marketable yield (kg·m⁻²): Total fresh fruit weight per plot at harvest
Water-use efficiency (WUE, kg·m⁻³): Calculated as yield/(irrigation/1000)
Nitrogen-use efficiency (NUE, kg fruit·kg⁻¹ N): Calculated as yield/(N applied/1000)

The Performance data is summarized in Table 4

TABLE 4
Performance Comparison - composition
powder vs. Control (Example 1)

| | Control | Soil Composition | 1% Change |
|---|---|---|---|
| Parameter | (mean ± SD) | (mean ± SD) | (95% CI) |
| Irrigation | 226.3 ± 18.0 | 118.9 ± 10.0 | −47.4% |
| (L · m⁻²) | | | (−51.2, −43.2) |
| Nitrogen Applied | 18.3 ± 1.0 | 14.9 ± 0.9 | −18.8% |
| (g · m⁻²) | | | (−22.9, −14.1) |
| Marketable Yield | 4.37 ± 0.12 | 5.19 ± 0.27 | +18.7% |
| (kg · m⁻²) | | | (+15.0, +23.0) |
| WUE | 19.4 ± 1.0 | 44.0 ± 4.8 | +126.9% |
| (kg · ⁻³) | | | (+112.7, +142.8) |
| NUE | 1239.7 ± 19.0 | 350.2 ± 20.0 | +46.1% |
| (kg fruit · kg⁻¹ N) | | | (+36.3, +55.3) |

Note:
Statistical Significance p < 0.001” with “p < 0.05 unless otherwise stated

### Key Findings:

1. Irrigation Reduction (500): The composition powder treatment reduced seasonal irrigation requirements by 47.4%, consistent with the range of 10-60% irrigation reduction as claimed herein. Despite receiving less than half the irrigation volume of control plots, composition powder-treated plants maintained comparable visual leaf turgor and chlorophyll content.
2. Yield Improvement (510): Marketable tomato yield increased by 18.7% in composition powder plots compared to control, achieving this improvement despite receiving 18.8% less nitrogen fertilizer. This yield increase falls within the claimed range of 15-30% yield improvement.
3. Water-Use Efficiency (520): WUE more than doubled (+126.9%) in composition powder-treated plots, demonstrating that the composition enabled substantially greater crop productivity per unit of irrigation water applied.
4. Nitrogen-Use Efficiency (530): NUE increased by 46.1% in composition powder plots, indicating improved retention and plant availability of applied nitrogen. The ability to achieve higher yields with reduced nitrogen input confirms the nutrient retention capabilities attributed to the zeolite component and improved soil structure created by the bentonite component.

### Statistical Analysis

Treatment effects were evaluated using one-way ANOVA with Welch's t-test for unequal variances (α=0.05). Bootstrap confidence intervals (95%) were generated using the bias-corrected and accelerated (BCa) method with 3,000 resamples. All measured differences between composition powder and control treatments were statistically significant at p<0.001, Welch's t-test, two-sided, α=0.05.

### Environmental Context

The 2024 growing season in Toronto experienced record-breaking precipitation (June-August total≈500 mm; July alone≈215 mm), representing the wettest summer on record for Toronto Pearson International Airport. To mitigate excessive rainfall impact, all plots were covered with transparent polyethylene sheeting on open-sided frames, allowing lateral airflow while reducing direct precipitation. The persistence of significant treatment effects under these challenging moisture conditions demonstrates the robustness of the composition's performance across variable environmental conditions.

### Discussion—Example 1

The results from Example 1 demonstrate that a two-component micronized mineral formulation (bentonite+zeolite) without biological enhancement can achieve substantial improvements in irrigation efficiency, nutrient-use efficiency, and crop yield. The 47.4% reduction in irrigation requirements while simultaneously achieving 18.7% higher yields represents a fundamental improvement in agricultural resource-use efficiency.

The mechanism of action is attributed to complementary mineral functions:
Bentonite (70 wt. %): Provides high swelling capacity (12.5 mL/2 g) and specific surface area (720 m²/g), enabling interlayer water retention and gradual release to plant roots
Zeolite (30 wt. %): Contributes selective cation exchange for NH₄⁺ and K⁺, reducing nutrient leaching while maintaining structural porosity

The micronized particle size distribution (D₅₀=12-15 μm) enabled:
1. Passage through standard 120-150 mesh fertigation filters without clogging
2. Intimate integration with soil particles to maximize soil-amendment contact
3. Short diffusion pathways for water and nutrient exchange
4. Substantially greater surface area (≥50-fold) compared to conventional granular amendments (D₅₀>850 μm)

### Example 2: Comparative Evaluation Including AMF Component (2025)

### Objective

To evaluate the independent and synergistic contributions of bentonite, zeolite, and arbuscular mycorrhizal fungi (AMF) to soil performance and crop productivity, and to demonstrate that the three-component formulation provides superior performance compared to two-component or single-component treatments.

### Experimental Design

Location: Toronto, Ontario, Canada
Soil Type: Garden loam, pH 6.4
Test Crop: Tomato (Solanum lycopersicum cv. ‘Roma’)
Total Area: 5 m² divided into 20 plots
Plot Size: 0.25 m² per plot (n=4 per treatment)
Growing Season: May-September 2025
   Treatment Groups (n=4 Plots Each):
1. Control (C): Untreated soil
2. Bentonite Only (B): 70% bentonite at 0.10 kg·m⁻²
3. Zeolite Only (Z): 30% zeolite at 0.043 kg·m⁻² (equivalent molar basis)
4. Bentonite+Zeolite (BZ) 70:30 blend at 0.10 kg·m⁻²
5. Bentonite+Zeolite+AMF (BZA) 70:30 mineral blend at 0.10 kg·m⁻² plus AMF inoculum applied in accordance with manufacturer specifications (see Example 2, AMF Application Protocol).

### Composition Specifications:

Mineral Components (identical to Example 1):
Micronized sodium bentonite: D₅₀=12 μm, D₉₀=35 μm
Micronized clinoptilolite zeolite: D₅₀=15 μm, D₉₀=38 μm

### AMF Component:

Species: Rhizophagus irregularis (syn. Glomus intraradices)
Form: Colonized root fragments and spores
Concentration: 5×10⁴ viable propagules per gram of inoculum
Application rate: 2.0 kg·ha⁻¹ (equivalent to 0.2 g·m⁻²)
Viability: 85% at time of application (confirmed by vital staining)

### Application Protocol

### Mineral Application:

Mineral components (B, Z, BZ, BZA treatments) were applied using the same protocol as Example 1: reconstitution at 1:40 (w/w) with high shear mixing, followed by manual application to achieve target dosing.

### AMF Application (BZA Treatment Only):

A two-stage protocol was employed to maximize AMF colonization:
Stage 1 (Soil Conditioning): The mineral fraction (bentonite+zeolite) was applied to soil 7 days prior to transplanting to allow soil structure modification and moisture equilibration.
Stage 2 (Root Inoculation): Immediately before transplanting, tomato seedling roots were dipped for 5 minutes in an AMF suspension prepared at 105 propagules. L⁻¹ in pH-buffered water (pH 6.5). Seedlings were then transplanted into the preconditioned soil plots.

This temporal separation of mineral conditioning and biological inoculation is consistent with our design to optimize both mineral integration and AMF establishment.

### Fertilization Protocol

To isolate the effects of the amendments, fertilization was standardized across treatment groups, with one exception:
Control: 18.0 g N·m⁻² (100% of soil-test recommendation)
All amended treatments (B, Z, BZ, BZA): 14.4 g N·m⁻² (80% of recommendation)

This design tests the hypothesis that mineral and biological amendments improve nutrient-use efficiency sufficiently to compensate for reduced fertilizer input.

### Measurements

Irrigation, nitrogen application, yield, WUE, and NUE were measured using protocols identical to Example 1.

Additional Measurement (BZA treatment only):
AMF Root Colonization: Root samples were collected at flowering stage (60 days after transplanting), cleared with 10% KOH, stained with trypan blue, and examined microscopically to confirm AMF colonization. Colonization percentage was quantified using the gridline-intersect method.

### Results

Performance data are summarized in Table 5.

TABLE 5
Comparative Performance Across Five Treatments (Example 2)

| | | | | | | % Irrigation |
|---|---|---|---|---|---|---|
| | Irrigation | N Applied | Yield | WUE | NUE | Reduction |
| Treatment | (L · m⁻²) | (g · m⁻²) | (kg · m⁻²) | (kg · m⁻³) | (kg · kg⁻¹ N) | vs. Control |
| Control (C) | 226.0 ± 12.0 | 18.0 ± 0.0 | 4.30 ± 0.20 | 19.0 ± 1.2 | 239.0 ± 12.3 | — |
| Bentonite (B) | 158.2 ± 8.0 | 14.4 ± 0.0 | 4.65 ± 0.25 | 29.4 ± 1.7 | 323.0 ± 20.1 | −30.0% |
| Zeolite (Z) | 202.8 ± 10.0 | 14.4 ± 0.0 | 4.70 ± 0.30 | 23.2 ± 1.6 | 326.0 ± 21.4 | −10.3% |
| BZ (Bentonite + | 124.3 ± 6.0 | 14.4 ± 0.0 | 5.20 ± 0.25 | 41.8 ± 2.9 | 361.1 ± 18.8 | −45.0% |
| Zeolite Composition) | | | | | | |
| BZA (Bentonite + | 101.5 ± 8.0 | 14.4 ± 0.0 | 5.65 ± 0.20 | 55.7 ± 3.1 | 392.4 ± 17.5 | −55.1% |
| Zeolite Composition + | | | | | | |
| AMF) | | | | | | |

### AMF Colonization Results (BZA Treatment):

Root colonization rate: 52±8% at 60 days after transplanting
Hyphal presence confirmed in rhizosphere soil samples
Arbuscule formation observed in cortical cells
No AMF colonization detected in control or mineral-only treatments

### Key Findings

### 1. Irrigation Reduction—Component Contributions:

Bentonite alone: 30.0% reduction
Zeolite alone: 10.3% reduction
Bentonite+Zeolite (BZ): 45.0% reduction
Bentonite+Zeolite+AMF (BZA): 55.1% reduction

The results demonstrate that bentonite (610) provides the dominant water-retention contribution due to its swelling capacity and high specific surface area, while zeolite (620) contributes more modestly to water retention. The combination of bentonite and zeolite (630) achieved approximately the sum of their individual contributions, while the addition of AMF (640) provided an additional 10 percentage point reduction beyond the mineral-only formulation. Synergy magnitude (~8 percentage points) was determined relative to additive expectation per ISO 10993-12 statistical comparison.

### 2. Synergistic Yield Enhancement:

### Comparing Yield Improvements Relative to Control:

Bentonite alone: +8.1%
Zeolite alone: +9.3%
BZ (two-component): +20.9%
BZA (three-component): +31.4%

The three-component formulation (740) achieved yield increases exceeding the predicted additive contributions of its components. If the effects were purely additive, BZ yield (730) would equal (B yield−C yield)+(Z yield−C yield)+C yield=4.76 kg m², whereas the observed BZ yield was 5.20 kg·m⁻² (+9.2% synergistic enhancement). All yield values expressed as mean±SD (n=4). The BZA (740) formulation further enhanced yield to 5.65 kg·m⁻², representing an additional +8.7% improvement attributable to AMF colonization.

### 3. Water-Use Efficiency:

### WUE Improvements Relative to Control:

B: +54.7%
Z: +22.1%
BZ: +120.0%
BZA: +193.2%

The BZA treatment achieved WUE of 55.7 kg m 3, representing nearly triple the WUE of untreated control plots, these differences were statistically significant at p<0.001 (Tukey HSD).

### 4. Nitrogen-Use Efficiency:

Despite receiving 20% less nitrogen than control, all amended treatments achieved higher NUE:
B: +35.1%
Z: +36.4%
BZ: +51.0%
BZA: +64.2%

The BZA treatment achieved the highest NUE (392.4 kg fruit kg-1 N), confirming that the tri-component system enables substantial fertilizer reduction without yield penalty.

### Statistical Analysis

Treatment effects were evaluated using one-way ANOVA followed by Tukey's HSD post-hoc test (α=0.05). All pairwise comparisons between treatments showed statistically significant differences (p<0.01) except for the B vs. Z comparison for yield (p=0.89), indicating that bentonite and zeolite provide similar modest yield benefits when applied individually.

The BZ (830) and BZA (840) treatments were significantly superior to all other treatments for all measured parameters (p<0.001).

### Key Mechanistic Insights:

1. Bentonite as the Primary Water-Retention Agent: The 30% irrigation reduction achieved by bentonite alone (vs. 10% for zeolite alone) confirms that sodium-activated swelling smectite is the dominant contributor to water-holding capacity enhancement, validating the emphasis on swelling properties.
2. Zeolite as the Primary Nutrient-Retention Agent: Despite its modest contribution to water retention, zeolite achieved similar yield improvements to bentonite (+9.3% vs. +8.1%), indicating its value for nutrient retention and slow release. The similar NUE improvements for B and Z treatments (+35% and +36%) further support zeolite's nutrient management role.
3. Mineral Synergy (BZ): The combination of bentonite and zeolite achieved 45% irrigation reduction, exceeding the sum of their independent contributions if applied sequentially (30%+10% would predict ~37% if additive, but 45% was observed). This ~8% enhancement represents synergistic interaction, likely due to bentonite creating micropores that retain water while zeolite maintains macroporosity for aeration and nutrient exchange. (910)
4. Biological Amplification (BZA): The addition of AMF to the mineral matrix provided an additional 10 percentage points of irrigation reduction (55% vs. 45%) and 8.7% yield enhancement beyond the BZ treatment. AMF colonization of 52% indicates successful establishment, with mycorrhizal hyphae extending the effective root exploration volume and accessing water and nutrients retained in the mineral matrix. The mineral components likely provided a protective and nutritive substrate supporting AMF spore germination, hyphal growth, and root colonization. (920)
5. Particle Size Importance: The consistent performance across both examples, using identical micronized particle specifications (D₅₀=12-15 μm), confirms that this particle size range is critical to achieving the claimed performance levels. The ability to deliver the formulation via fertigation without filter clogging (demonstrated by successful use of 120-150 mesh filters) validates the importance of particle size specifications for fertigation compatibility.

### Example 3: Particle Size Comparison—Micronized vs. Granular (Theoretical Calculation)

### Objective

To quantify the specific surface area advantage of the micronized formulation (D₅₀=10 μm) compared to conventional granular amendments (D₅₀=0.85-2 mm) referenced in prior art.

### Calculation

### Assumptions:

Spherical particle approximation
Bentonite density: 2.4 g·cm⁻³
Zeolite density: 2.2 g·cm⁻³

### Specific Surface Area Calculation:

For spherical particles: SSA=6/(ρ×d), Formula derived from geometric approximation for monodisperse spheres; non-spherical corrections ≤10%

Where:
SSA=specific surface area (m²·g⁻¹)
ρ=particle density (g·m⁻³)
d=particle diameter (m)

### Micronized Formulation (D₅₀=10 μm Average):

Bentonite: SSA=6/(2.4×0.001)=2,500 m² g⁻¹ (geometric)
Zeolite: SSA=6/(2.2× 0.001)=2,727 m²·g⁻¹ (geometric)
Blend (70:30): SSA=2568 m²·g⁻¹ (geometric)

Note: Actual measured SSA for swelling smectite (720 m²·g⁻¹) includes internal surface area from interlayer spaces, not just external geometric surface.

### Conventional Granular (D₅₀=1 mm):

Bentonite: SSA=6/(2.4×0.1)=25 m²·g⁻¹ (geometric)
Zeolite: SSA=6/(2.2× 0.1)=27 m² g⁻¹ (geometric)
Blend (70:30): SSA=25.6 m²·g⁻¹ (geometric)

### Surface Area Ratio:

Micronized/Granular=2,568/25.6=approximately 100-fold (≥75-fold as claimed) greater geometric surface area

This calculation confirms that the micronized formulation provides specific surface area improvements of at least 75-fold, with actual values approaching 100-fold for external geometric surface area. When internal surface area of swelling smectite is considered, total accessible surface area differentials exceed 100-fold.

### Practical Implications:

1. The 74-fold surface area increase enables proportionally faster water adsorption kinetics
2. Greater soil-particle contact area per unit weight of amendment
3. Shorter diffusion pathways for nutrient exchange
4. Enhanced interfacial interactions with AMF hyphae

This substantial surface area advantage directly contributes to the observed 5-10-fold improvement in water retention capacity per unit weight and superior distribution uniformity throughout the soil profile.

### Comparative Summary—Examples 1 & 2

Table 6 provides a comparative summary of the key performance metrics from both Toronto field trials.

TABLE 6
Summary of Field Performance Data

| | Example 1 | Example 2 | Example 2 |
|---|---|---|---|
| | (2024) BZ | (2025) BZ | (2025) BZA |
| Parameter | vs. Control | vs. Control | vs. Control |
| Irrigation Reduction | 47.4% | 45.0% | 55.1% |
| Yield Increase | 18.7% | 20.9% | 31.4% |
| WUE Improvement | 126.9% | 120.0% | 193.2% |
| NUE Improvement | 46.1% | 51.0% | 64.2% |
| Fertilizer Input | 18.8% | 20.0% | 20.0% |
| Reduction | | | |
| AMF Colonization | Not applicable | Not applicable | 52% |

### Consistency Across Trials:

The close agreement between Example 1 (2024) and the BZ treatment in Example 2 (2025) confirms reproducibility of the mineral-only formulation performance:
Irrigation reduction: 47.4% vs. 45.0% (within 2.4 percentage points)
Yield increase: 18.7% vs. 20.9% (within 2.2 percentage points)

The additional performance enhancement provided by AMF (BZA treatment) represents an incremental improvement of approximately:
+10 percentage points irrigation reduction beyond BZ
+10 percentage points yield increase beyond BZ
+73 percentage points WUE increase beyond BZ
+13 percentage points NUE increase beyond BZ

These incremental improvements attributable to AMF colonization validates the value of the three-component system.

These examples collectively establish that the claimed composition and methods deliver quantifiable, reproducible, and commercially significant improvements in agricultural resource-use efficiency while maintaining or enhancing crop productivity.

### 9. Advantages Over Prior Art

The disclosed TTC (Tri-Technology Composition) and associated methods represent a significant advancement over prior soil amendment technologies, as evidenced by quantified irrigation and yield improvements reported herein. By integrating physical (smectite clay), chemical (zeolite), and biological (AMF) functionalities, the formulation overcomes multiple limitations inherent to conventional single-component or non-adaptive solutions.

The table below (Table 7) summarizes key limitations in the prior art and the corresponding advantages of the disclosed invention:

TABLE 7
Key limitations in the prior art and solutions of the invention

| Limitation of Prior Art | Solution |
|---|---|
| Single-function amendments (e.g., | Integrates smectite (water retention), zeolite (nutrient |
| clay-only, zeolite-only, AMF-only) | retention), and AMF (biological enhancement) for a holistic, |
| | multi-mechanism approach |
| Coarse particle size (>50 μm) limits | Uses micronized particles (D₅₀ = 1-15 μm) for improved |
| surface area, soil dispersion, and | reactivity, even soil distribution, and compatibility with |
| equipment compatibility | fertigation and spray systems including 120-mesh filters as |
| | tested under ASTM D 422 soil texture standards. |
| AMF inoculants lack structural | Mineral fraction pre-conditions the soil, improving |
| support in the soil environment | aggregation and colonization substrate for AMF; two-stage |
| | protocol maximizes AMF viability |
| Fixed, one-size-fits-all formulations | Adaptive system selects component ratios based on soil |
| ignore local soil constraints | diagnostics, targeting specific limitations (e.g., water scarcity, |
| | nutrient leaching, low organic matter) |
| Incompatibility of microbial and | Controlled pH (5.5-7.5), ionic strength (≤50 mM), low-shear |
| mineral components in storage or | mixing, and optional staged addition ensure microbial |
| application | viability over 3-6 months |
| Limited application formats (e.g., | Offered in multiple formats (dry powder, granulated, liquid |
| granular-only, liquid-only) | concentrate, two-part kit), enabling use with broadcast, spray, |
| | fertigation, and in-furrow methods. Packaging materials and |
| | shelf-life stability ≥6 months verified under ambient storage |
| Use of synthetic soil conditioners | Composed of natural minerals and biologicals; enables 20- |
| (e.g., polyacrylamide) or high | 50% irrigation reduction and 10-30% fertilizer reduction |
| fertilizer input carries environmental | while maintaining or increasing yield |
| costs | |
| Poor performance of individual | The synergistic tri-component blend achieves effective soil |
| components in severely degraded or | conditioning even in soils where individual components, |
| marginal soils | when applied separately, fail to produce measurable irrigation |
| | or yield benefits (see Examples 1-2). |

1. A soil-conditioning composition comprising, in admixture:
(i) a swelling smectite clay having a median particle size (D₅₀) of 1 to 50 micrometers and a 90th-percentile particle size (D₉₀) of no more than 100 micrometers, wherein the smectite exhibits interlayer expansion upon hydration;
(ii) a zeolite having a median particle size (D₅₀) of 1 to 50 micrometers and a D₉₀ of no more than 100 micrometers;
(iii) viable arbuscular mycorrhizal fungi (AMF) propagules at a concentration of 10³ to 10⁷ propagules per gram;
(iv) wherein the composition comprises a swelling smectite clay in an amount of 10 to 90 percent by weight, zeolite in an amount of 10 to 90 percent by weight, and arbuscular mycorrhizal fungi in an amount of 0.05 to 20 percent by weight, relative to total solids;
(v) wherein the composition further comprises 0 to 10 weight percent humic substances and/or 0 to 2 weight percent hydrocolloid stabilizer;
(vi) wherein the composition is reconstituted with water at a weight-to-weight ratio of 1:20 to 1:50 to yield a homogeneous suspension having a pH of 5.5 to 7.5 and an ionic strength not exceeding 50 mM; and
(vii) wherein the composition, when reconstituted as described, passes through a 120-mesh filter without observable agglomeration or nozzle obstruction under standard fertigation conditions.

2. The composition of claim 1, wherein the smectite clay exhibits a swelling capacity of at least 10 mL per 2 g upon hydration, with interlayer expansion driven by hydrated cations selected from sodium (Na⁺), calcium (Ca²⁺), or combinations thereof, as determined by ASTM D5890 or an equivalent standardized method.

3. The composition of claim 1, wherein the smectite comprises sodium bentonite, sodium-activated bentonite, sodium-activated hectorite, or sodium-activated saponite; the zeolite comprises clinoptilolite, chabazite, or mordenite; and the AMF comprises spores, colonized root fragments, or hyphae selected from Rhizophagus irregularis, Funneliformis mosseae, Claroideoglomus etunicatum, Rhizophagus clarus, or equivalents thereof.

4. The composition of claim 1, wherein the relative weight percentages of smectite clay, zeolite, and arbuscular mycorrhizal fungi are selected according to soil diagnostic characteristics such that:
(i) for sandy soils having sand content ≥70 percent, 60 to 80 weight percent smectite, 20 to 35 weight percent zeolite, and 2 to 5 weight percent fungi;
(ii) for soils with cation-exchange capacity (CEC)<10 cmol(+)/kg, 30 to 40 weight percent smectite, 50 to 70 weight percent zeolite, and 1 to 3 weight percent fungi;
(iii) for saline soils having electrical conductivity (EC)>4 dS/m, 35 to 45 weight percent smectite, 35 to 45 weight percent zeolite, and 3 to 6 weight percent fungi; or
(iv) for degraded soils having organic-matter content <1 percent, 35 to 45 weight percent smectite, 35 to 45 weight percent zeolite, and 5 to 10 weight percent fungi.

5. The composition of claim 1, wherein the combination of smectite, zeolite, and AMF provides a synergistic improvement beyond the additive effects observed when any two components are used independently, the improvement being characterized by:
(i) an increase in soil water-holding capacity of at least 10 percent relative to the sum of effects observed when each component is used independently or in binary combinations;
(ii) a reduction in fertilizer requirements of at least 10 percent compared to any two-component or single-component control; and
(iii) a crop-yield increase of at least 10 percent relative to such controls.

6. The composition of claim 1, wherein the smectite clay and the zeolite together provide a specific surface area at least 75 times greater than that of comparable granular particles having sizes greater than 500 micrometers, as measured by BET nitrogen adsorption in accordance with ASTM D3663, while avoiding classification as a nanomaterial by maintaining a minimum particle size greater than 1 micrometer.

7. The composition of claim 1, wherein at least 90 percent of the particles fall within 1 to 50 micrometers, not more than 5 percent exceed 100 micrometers, not more than 2 percent are smaller than 1 micrometer, and AMF viability is at least 80 percent post-processing as determined by vital-staining assay.
Kit (Independent, Article of Manufacture)

8. A kit comprising:
(i) the composition of tailor-made soil amendment;
(ii) a first container including micronized smectite and zeolite having a median particle size (D₅₀) of 1 to 50 micrometers and a 90th-percentile particle size (D₉₀) of no more than 100 micrometers;
(iii) a second container including arbuscular mycorrhizal fungi (AMF) propagules at a concentration of 10³ to 10⁷ propagules per gram; and
(iv) instructions for high-shear mixing of the first component at 500 to 1 500 rpm followed by low-shear incorporation of the second component at less than 300 rpm;
wherein the kit is adapted for reconstitution in accordance with the composition parameters.
Method of Application/AI Optimization (Independent)

9. A method of conditioning soil, the method comprising:
(i) measuring at least two distinct soil-diagnostic parameters selected from the group consisting of soil texture, cation-exchange capacity (CEC), electrical conductivity (EC), organic-matter (OM) content, available-water capacity (AWC), pH, and combinations thereof, wherein said parameters may further include any measurable soil property determined by analytical, sensor-based, imaging, spectroscopic, computational, or equivalent diagnostic techniques;
(ii) selecting a formulation ratio of the composition of claim 1 based on predefined decision rules or a computer-implemented agricultural-management tool configured to control physical dosage;
(iii) preparing a composition according to claim 1 using the selected recommendation; and
(iv) applying the composition via fertigation through irrigation systems with 80 to 200-mesh filtration or spraying.

10. The method of claim 9, wherein the composition of claim 1 is applied using a technique selected from spray application at 40 to 70 m³/ha, in-furrow injection at 8 to 25 m³/ha, broadcast application followed by incorporation to a depth of 5 to 30 cm, or delivery as a slurry via drip irrigation, thereby producing at least one of:
(i) a 10 to 50 percent increase in water retention;
(ii) a 1 to 10 cmol(+)/kg increase in CEC;
(iii) a 10 to 50 percent increase in microbial biomass; or
(iv) a 0.1 to 0.5 percent increase in organic-matter content over 2 to 5 years; and achieving at least one of:
(i) a 10 to 60 percent reduction in irrigation volume;
(ii) a 10 to 30 percent reduction in fertilizer requirements; or
(iii) a 15 to 30 percent increase in crop yield relative to untreated control, as measured under field conditions.

11. The method of claim 9, further comprising a two-stage application wherein:
(i) Stage 1 comprises applying the mineral fraction (smectite and zeolite) to the soil 1 to 30 days prior to planting; and
(ii) Stage 2 comprises applying the AMF propagules at planting via root-dipping or in-furrow application,
wherein such temporal separation increases AMF-root colonization by at least 20 percent, measured using gridline-intersect microscopy.

12. The method of claim 9, wherein the composition is applied to degraded soil exhibiting at least one property selected from:
(i) sand content of at least 70 percent;
(ii) cation-exchange capacity (CEC) of 6 cmol(+)/kg or less;
(iii) electrical conductivity (EC) greater than 4 dS/m; or
(iv) organic-matter content below 1 percent;
using a technique selected from:
(a) spray application at 40 to 70 m³/ha;
(b) in-furrow injection at 8 to 25 m³/ha;
(c) broadcast incorporation to a depth of 5 to 30 cm; or
(d) drip-irrigation slurry application;
at a rate of 200 to 2 000 kg/ha, wherein said application results in:
(i) a 10 to 50 percent increase in water retention;
(ii) a 1 to 10 cmol(+)/kg increase in CEC;
(iii) a 10 to 50 percent increase in microbial biomass; and
(iv) a 0.1 to 0.5 percent increase in organic-matter content over 2 to 5 years.

13. The method of claim 9, wherein the formulation ratio is selected via an algorithm implemented on a computer-readable medium, the algorithm comprising:
(i) accessing a database comprising soil-diagnostic data, formulation records, and performance metrics across multiple sites and seasons;
(ii) executing a machine-learning model trained to predict optimal formulation ratios and application rates;
(iii) applying a feedback loop configured to update predictive outputs based on observed field performance;
(iv) providing, through a user interface, site-specific recommendations including predicted agronomic outcomes and associated confidence levels; and
(v) incorporating, within the database and predictive model, additional soil-amendment components beyond smectite, zeolite, and AMF, including but not limited to biochar, humic or fulvic substances, mineral or organic conditioners, and functionally equivalent or successor materials identified through ongoing research.

14. The method of claim 9, wherein the machine-learning algorithm further optimizes the formulation ratio and recommended application parameters based on a cost function that accounts for input-material costs, irrigation energy, user-defined priorities, and fertilizer usage to minimize total cultivation expense while maintaining agronomic performance.
Comparative and Environmental Performance (Independent)

15. A method of improving soil and crop performance, the method comprising applying the composition of claim 1 to a target soil at a rate of 200 to 2 000 kg/ha, wherein the composition yields performance improvements exceeding those of:
(i) granular formulations comprising smectite and zeolite with or without AMF;
(ii) micronized minerals without AMF; and
(iii) AMF formulations lacking a mineral matrix.

16. The method of claim 15, wherein compared to granular formulations, the composition provides:
(i) at least two-fold greater water retention per unit weight as determined by gravimetric soil-moisture test (ASTM D2216);
(ii) at least five-fold improved distribution uniformity; and
(iii) compatibility with drip-irrigation systems using 120 to 200-mesh filters.

17. The method of claim 15, wherein the composition provides:
(i) at least 10 percent greater crop yield;
(ii) at least two-fold greater AMF-root colonization versus AMF-only treatments; and
(iii) at least 10 percent greater nutrient-use efficiency versus either component alone.

18. The method of claim 9, wherein the algorithm or decision-support system is integrated with a soil-moisture or nutrient-sensor network that automatically adjusts irrigation or fertigation parameters in real time according to predicted soil-amendment performance and environmental feedback.

19. The method of claim 15, wherein application of the composition reduces greenhouse-gas emissions from soil respiration or fertilizer use by at least 10 percent relative to untreated control plots, as determined in accordance with ISO 14064 or an equivalent carbon-accounting methodology.

20. The method of claim 9, wherein the algorithm operates within a closed-loop adaptive management system that:
(i) integrates real-time soil and climatic data from satellite, drone, or in-situ sensor networks;
(ii) continuously refines the formulation ratio and application parameters through reinforcement learning based on multi-season agronomic performance;
(iii) predicts site-specific soil carbon sequestration potential and water-retention gains using spatiotemporal models;
(iv) autonomously adjusts future application schedules to maintain optimal soil health and yield metrics under changing climatic or economic conditions; and
(v) generates verifiable sustainability and carbon-accounting reports compatible with international certification frameworks including ISO 14064 and FAO's Global Soil Partnership protocols.
